(19) United States
(12) Patent Application Publication
(10) Pub. No.: US 2015/0297064 A1
(43) Pub. Date: Oct. 22, 2015

(54) ENDOSCOPE GUIDE TUBE

(71) Applicant: ENDOCOLE, LLC, Santa Barbara, CA (US)

(72) Inventors: Jeff VASEK, Santa Barbara, CA (US); Steven RYCHNOVSKY, Santa Barbara, CA (US)

(21) Appl. No.: 14/367,113

(22) PCT Filed: Dec. 19, 2012
(86) PCT No.: PCT/US2012/070661

§ 371 Date: Jun. 19, 2014

Related U.S. Application Data
(60) Provisional application No. 61/577,608, filed on Dec. 19, 2011.

(51) Int. Cl. A61B 1/00

(52) U.S. Cl. CPC A61B 1/00151; A61B 1/00154

(57) ABSTRACT

Described herein are endoscopic guide tube devices, systems and methods for using the guide tubes to facilitate insertion of an endoscope into a body cavity. The device may include a pressurization chamber having a nozzle, an everting tube mounted within the pressurization chamber, and a valve located at or proximate the distal end of the everting tube. The valve can be configured to open when the everting tube is fully deployed. A tether can be attached to the distal end of the everting tube to restrain the everting tube from fully deploying until a release mechanism is engaged. The valve can be formed from two sheets that are adjacent to each other and/or can include a viscous gel.

### CROSS REFERENCE TO RELATED APPLICATIONS

This application claims the benefit of U.S. Provisional Application No. 61/577,608 filed Dec. 19, 2011 titled “Endoscope Guide Tube” which is herein incorporated by reference in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### FIELD

The invention relates generally to guide tubes that can be inserted into a body cavity. More particularly, the invention relates to guide tubes that can be used in endoscopic procedures.

### BACKGROUND

In medical procedures it is often necessary to place a medical device within a body lumen. Examples here include colonoscopy, endoscopy, arthroscopic procedures, catheterization and the like. In such procedures it is desirable to limit inadvertent damage to the body lumen that can be caused by the medical device. Similarly, it can be difficult to place with device within the body lumen due to the many branches and folds that often exist in such body lumens. As a consequence, it is often desirable to first place a guide tube or lumen within the body lumen itself. Such a tube can limit body damage and speed up the medical procedure, i.e. the physician's efforts can be focused on applying the medical device rather than placing it. For example, before performing a colonoscopy it is first necessary to insert and place a relatively stiff colonoscope into the colon. Safe placement requires that the physician must carefully insert and advance the colonoscope over 1 meter into the colon without causing mechanical damage to the colon itself. As a consequence, the individual inserting the colonoscope often requires years of training and practice. While experienced physicians have low injury rates, the training required and the time added to the colonoscopy procedure result in higher costs and added procedure time.

One means of circumventing these requirements is to first place a guide tube within the body lumen. If such a guide tube were cost-effective and easily deployed, it could yield a safer procedure in which a guide tube is first deployed within the colon, after which the colonoscope can be rapidly and safely placed. Ideally, deployment of such a guide tube could be done with ease and provide little risk such that it could either be deployed by a less experienced operator or it could be more rapidly deployed by an experienced operator. Such a device would reduce the time that the skilled physician must spend with the patient and lower the overall cost of the procedure. Unfortunately, no guide tube schemes disclosed to date have met these criteria and, as a consequence, they have not been widely adopted.

In most schemes disclosed in the prior art, the guide tube is deployed within the body lumen by eversion with a means of pressurization provided at the proximal end. A major limitation of the prior art is associated with the high resistive forces that occur as the guide tube is deployed. Such resistive forces can arise during guide tube deployment as a result of twisting of the guide tube or as a result of the general tendency for the guide tube to become tangled or disordered at its proximal end. Resistive forces can also arise when air pockets are trapped within the center region of the guide tube so that the inverted section of the guide tube is not fully compressed. This increases the cross sectional area of the guide tube and provides a larger inner surface area, thereby increasing the overall resistive drag force associated with this surface. Furthermore, the prior art either uses no distal valve to limit outward fluid flow or uses valve schemes that are overly cumbersome or ineffective. As a result of these and similar restrictions, the systems used in the prior art require deployment pressures and inflation fluid volumes that are higher than desirable and that pose a risk of bodily injury. Furthermore, most systems in the prior art are overly cumbersome and impractical to operate. As a consequence, systems disclosed in the prior art are not in widespread use today.

### SUMMARY OF THE INVENTION

The present invention relates generally to guide tubes that can be inserted into a body cavity. More particularly, the invention relates to everting guide tubes that can be used in endoscopic procedures.

In some embodiments, an endoscopic guide tube device to facilitate the insertion of an endoscope or similar device into a body lumen is provided. The device can include a pressurization chamber comprising a nozzle and an inner surface of a wall that defines an interior, wherein the nozzle has an opening; an everting tube having a proximal end and a distal end, wherein the everting tube is mounted within the pressurization chamber with the proximal end of the everting tube attached to the pressurization chamber; a port on the pressurization chamber configured to allow a positive pressure to be delivered to the interior of the pressurization chamber, wherein the positive pressure is configured to deploy the everting tube outwards through the nozzle; and a valve located at or proximate the distal end of the everting tube, wherein the valve is configured to open when the everting tube is fully deployed.

In some embodiments, an endoscopic guide tube device to facilitate the insertion of an endoscope or similar device into a body lumen is provided. The device can include a pressurization chamber containing an everting tube mounted within said pressurization chamber with a proximal end of said everting tube mounted to be attached to said pressurization chamber; a port on said pressurization chamber whereby a positive pressure can be delivered to the interior of said pressurization chamber to cause said everting tube to deploy outward through said nozzle; and a self-sealing valve means located at or near the distal end of said everting tube which opens when guide tube is fully everted.

In some embodiments, the pressurization chamber of the endoscopic guide tube device has a pressure relief valve configured to prevent excessive positive pressures from occurring within the everting tube. In some embodiments, the pressure relief valve is configured to prevent excessive positive pressures from occurring within the everting tube during or after deployment. In some embodiments, the pressurization chamber is in communication with a pressure sensor configured to measure pressure within the pressurization chamber.

In some embodiments, the everting tube is wound on a deployment spool located within the pressurization chamber. In some embodiments, the deployment spool comprises a central axle and an outer diameter surface on which the everting tube is wound. In some embodiments, the deployment spool has a diameter equal or greater than 0.2 inches. In some embodiments, the central axle of the deployment spool is positioned such that a portion of the outer diameter surface of the deployment spool is nominally aligned with the opening of the nozzle to reduce or minimize frictional resistance between the everting tube and the nozzle during deployment of the everting tube.

In some embodiments, the pressurization chamber has restraints or indentations on opposite sides of the inner surface of the pressurization chamber which are configured to restrain the central axle of the deployment spool, wherein the restraints or indentations do not extend through the wall of the pressurization chamber such that the deployment spool axle is fully contained within the pressurization chamber and does not extend through the wall of the pressure chamber. In some embodiments, either one end or both ends of the central axle of the deployment spool extends through the inner surface of the pressurization chamber, wherein a wheel is attached to one end of the axle or wheels are attached on both ends of the axle, wherein the one or more wheels are located outside the pressurization chamber and are configured to apply an external twisting force to the deployment spool. In some embodiments, the device further includes an axle extended across the interior of the pressurization chamber, wherein the deployment spool comprises a cylindrical tube disposed over the axle such that the deployment spool can rotate independently of the axle. In some embodiments, the device further includes an axle extended between the two sides of the pressurization chamber and the spool is formed by a cylindrical tube that passes over the axle but where the spool is not rigidly attached to the axle and may rotate independently of the axle. In some embodiments, the deployment spool comprises a hollow shaft with no axle that is located inside the pressurization chamber and is constrained within the pressurization chamber by restraints located on opposite walls of the pressurization chamber.

In some embodiments, the everting tube is attached to the nozzle and the nozzle is detachable from the pressurization chamber to allow the nozzle and attached everting tube to be physically separated from the pressurization chamber. In some embodiments, the nozzle incorporates a seal such that when the endoscope is inserted through the nozzle the seal resists fluid escape from around the endoscope where the endoscope enters the nozzle. In some embodiments, the nozzle incorporates a seal such that when the endoscope is inserted through the nozzle the seal resists fluid escape from the space between the inside surface of the nozzle and the outside surface of the endoscope where it enters the nozzle. In some embodiments, a second port is located on the nozzle to allow fluid communication to the interior of the everted tube, wherein the second port is configured to allow application of positive pressure to the interior of the everted tube from an external pressure source. In some embodiments, the nozzle has a distal end that is comprised of a non-rigid elastomeric material. In some embodiments, the nozzle has a proximal end, a distal end and a central region between the proximal end and the distal end, wherein the outside diameter of the central region of the nozzle is smaller than both the outside diameter at the proximal end and the outside diameter at the distal end of the nozzle. In some embodiments, the nozzle is formed from two or more mated pieces that can be separated to allow removal of the nozzle from the endoscope without having to remove the endoscope from within the nozzle.

In some embodiments, the everting tube has a seal at its proximal end that mates with the nozzle, wherein the seal is configured to allow the everting tube to be separated from the nozzle such that the endoscope can then be inserted into the proximal end of the everting tube through the seal.

In some embodiments, prior to deployment, the everting tube is folded within the pressurization chamber in an accordion-like manner into a plurality of folds such that the orientation of the folds within the pressurization chamber is orthogonal to the orientation of the central axis of the everting tube after deployment. In some embodiments, prior to deployment, the everting tube is wrapped within the pressurization chamber in a looped manner to form loops with an alternating winding orientation such that when the everting tube is deployed the twisting of the everting tube along its axis is reduced. In some embodiments, the everting tube is wrapped within said pressurization chamber in a looped manner such that the everting tube is twisted about +180 degrees around its axis and held in place and then twisted about −180 degrees about its axis and then held in place such that when said everting tube is deployed the net result will be little or no residual twisting of said everting tube along its axis. In some embodiments, the loops form a stacked arrangement within the pressurization chamber with the central axis of the stacked loops being generally parallel to the alignment of the everting tube when fully deployed. In some embodiments, the loops formed from the twisted everting tubes are stacked in an alternating manner such that the final arrangement forms a figure eight pattern with two sets of stacked loops, each of whose central axes are generally aligned parallel with the axis of the everting tube when fully deployed.

In some embodiments, the everting tube is formed from extruded low durometer polyurethane. In some embodiments, the wall thickness of the everting tube is 0.010 inches or less. In some embodiments, the everting tube is rolled or folded along its axial length prior to wrapping or folding it within the pressurization chamber. In some embodiments, the everting tube comprises a single material formed in a manner such that the everting tube has a section having a first diameter followed by a section having a second diameter which is repeated along the length of the everting tube to form an undulating pattern or a ribbed pattern along at least one half the length of the everting tube. In some embodiments, the surface of the everting tube is modified to reduce either its coefficient of friction or its adhesion properties, wherein the modification is selected from the group consisting of plasma energy treatment, application of polyvinylypyrrolidone, application of hyaluronic acid, application of parylene, application of friction reducing surface treatments, application of a biocompatible lubricating agent, application of glycerin, application of propylene glycol, application of a hydrophobic silicone based lubricant, and application of a water based lubricant.

In some embodiments, the distal end of the everting tube is temporarily sealed prior to deployment and wherein the distal end of the everting tube is configured to be opened by applying a force to the temporary seal at the distal end of the tube that is greater than the force used to cause the everting tube to deploy from the pressurization chamber. In some embodiments, the distal end of the everting tube is closed and wherein the distal end of the everting tube is configured to be opened by cutting or puncturing.

In some embodiments, the valve is formed from or attached to the distal end of the everting tube that has been modified such that, prior to full eversion, the distal end of the everting tube comprises two flat sections that are connected along two edges and have two common surface faces that are generally in direct contact with each other such that there is no significant gap between the two flat sections including at the edges where the two flat sections are connected such that a fluid barrier exists that prevents fluid from entering into the distal end of the everting tube. In some embodiments, the valve is integral to the distal end of the everting tube. In some embodiments, the valve comprises a distal portion of the everting tube that is temporarily sealed by an application of a viscous gel or grease on the interior surface of the everting tube. In some embodiments, the valve comprises an elastomeric material connected to the distal end of the everting tube, wherein in an unpressurized state the elastomeric material is in a constricted state that seals the distal end of the everting tube. In some embodiments, the valve is configured to open at an applied pressure of 3 to 5 psi.

In some embodiments, a method of inserting an endoscope into a body lumen is provided. The method can include providing an everting tube having a proximal end and a sealed distal end; partially deploying the everting tube within the body lumen; advancing the endoscope into the partially deployed everting tube while maintaining a nominal seal between the endoscope and the proximal end of the partially deployed everting tube; advancing the endoscope within the everting tube until the endoscope reaches the distal end of the partially deployed tube; applying pressure to the sealed distal end using the endoscope; and opening the sealed distal end to fully deploy the everting tube.

In some embodiments, the method further includes inflating the everting tube via pressurization delivered from the endoscope. In some embodiments, the method further includes inflating the everting tube through a port in communication with the everting tube using an external pressure source.

In some embodiments, an endoscopic guide tube device to facilitate the insertion of an endoscope or similar device into a body lumen is provided. The device can include a pressurization chamber comprising a nozzle and an inner surface that defines an interior, wherein the nozzle has an opening; an everting tube having a proximal end and a distal end, wherein the everting tube is mounted within the pressurization chamber with the proximal end of the everting tube attached to the pressurization chamber; a port on the pressurization chamber configured to allow a positive pressure to be delivered to the interior of the pressurization chamber, wherein the positive pressure is configured to deploy the everting tube outwards through the nozzle; and a tether having a proximal end and a distal end, wherein the distal end of tether is attached to one or more locations on a distal portion of the everting tube, wherein the proximal end of the tether is separately connected or held to an external body such that, prior to full deployment of the everting tube, the length of the distance between the two ends of the tether is less than the fully deployed length of the everting tube such that the tether prevents full deployment of the everting tube until the tether is released from the external body.

In some embodiments, the device can further include a distal seal located at the distal end of the everting tube. In some embodiments, the distal seal comprises two sheets connected at the edges and lying adjacent to each other with no space existing between the two sheets. In some embodiments, the distal seal comprises a viscous or adhesive material disposed within the distal end of the everting tube. In some embodiments, the distal seal comprises a pressure bond at the distal end of the everting tube. In some embodiments, the distal seal comprises an elastic or non-elastic material disposed around the distal end of the everting tube. In some embodiments, the distal seal comprises a portion of the tether wrapped around the distal end of the everting tube.

In some embodiments, the device can further include a deployment spool located within the pressurization chamber, wherein the proximal end of the tether passes though the nozzle of the pressurization chamber and is releasably attached to the deployment spool.

In some embodiments, the external body is dimensioned larger than either the inner diameter of the nozzle or the everting tube such that the external body is too large to pass through the nozzle.

In some embodiments, the valve is self-sealing.

In some embodiments, the device further includes a second port configured to allow negative pressure to be applied to the interior of the everted tube while a positive pressure is applied within the interior of the pressurization chamber to create two forces which both act to remove trapped air located within the everting tube before it is deployed.

In some embodiments, the device further includes a temporary seal placed near the proximal end of the everting tube prior to deployment and a second temporary seal placed at the distal end of the everting tube prior to deployment, wherein the temporary seal at the proximal end is physically accessible via the nozzle opening such that a negative pressure can be applied to the interior of the everting tube such that residual gas or fluid can be removed to reduce the cross sectional area of the everting tube.

In some embodiments, the device further includes a temporary seal located on the distal end of the everting tube and a second removable seal located over the nozzle opening to provide an airtight seal such that a negative pressure to remove air can be applied to an interior volume formed by the seals and the everting tube.

In some embodiments, the everting tube comprises a wall structure that includes a first flexible material and a second flexible material that has a higher modulus of elasticity than the first material used to form the everting tube wall, wherein the second material is wrapped around the first material in loops or a helical pattern.

In some embodiments, a semi-rigid tube device is provided. The device can include a tube having an outside diameter smaller than the everting tube and an inside diameter larger than the endoscope with openings at both ends and a flange at its base to prevent passage through the rectum. In some embodiments, the tube is flexible and has a slit that runs along its entire length where the tube material is a polymer that is sufficiently flexible such that when an endoscope is passed through the semi-rigid tube, the semi-rigid tube can be pulled sideways off the endoscope by allowing the endoscope to pass through the slit.

In some embodiments, a method of performing endoscopy in the colon is provided. The method can include deploying an everting guide tube within the colon then inserting the semi-rigid tube into the everting guide tube. The everting guide tube and/or the semi-rigid tube can be advanced past the sigmoid flexure of the colon to straighten the colon, after which an endoscope is advanced into the colon.

In some embodiments, the semi rigid tube device is employed and the method adds the additional step of retracting the semi-rigid tube after the endoscope has been inserted and removing the semi-rigid tube by pulling it sideways off the endoscope by passing the endoscope through the slit in the side of the semi-rigid tube.

In some embodiments, a method of manufacturing the device is provided. The method can include applying a negative pressure to the interior of the everting tube before it is wrapped or folded.

In some embodiments, the method further includes placing a temporary seal near the proximal end of the everting tube and placing a second temporary seal at the distal end of the everting tube by either compression and wrapping of the everting tube or by placement of a temporary seal around the outside of the everting tube, after which a needle and syringe or similar device is inserted through either the temporary seal and a negative pressure is applied to the interior volume within the sealed everting tube in order to reduced the cross sectional area of the everting tube. In both cases the temporary seals open when pressure is applied to the pressurization chamber to cause deployment of the everting tube.

In some embodiments, the device is formed by heating and pressing a section of tube having an otherwise nominally circular cross section such that the material is heated to at or near its transition temperature such that its shape is permanently changed from a nominally circular cross section to a nominal cross section existing substantially of two flat sheets connected along their two edges.

In some embodiments, a method of manufacturing the device is provided. The method can include providing a thin flat material having a width nominally equal to 3.14*D/2, where D is the tube diameter, and inserting the thin flat material into the distal end of the everting tube prior to heating in order to prevent the two surfaces from bonding together.

In some embodiments, a method of manufacturing the device is provided. The valve can be formed by bonding two flat sheets to the distal end of a nominally circular tube and bonding those two sheets together along their edges so that the distal end of the tube terminates in a normally closed, flat valve. In some embodiments, the valve can be formed by applying a viscous gel or grease on the interior surfaces of a nominally circular everting tube near its distal end to temporarily seal the end of the tube until it is fully deployed by eversion delivery. In some embodiments, a viscous grease or gel substance is applied on the internal surfaces of the everting tube at its distal end within the formed valve region the evertable tube.

In some embodiments, a passive valve that is normally closed is provided. The valve can be located at the distal end of a nominally circular evertable tube wherein the valve includes a deformation of the nominally circular evertable tube within its distal region end region such that when the tube is in its relaxed, non-pressurized state, the deformed section forms a shape that substantially comprises two flat sheets connected at the edges and lying adjacent to each other with no space existing between the two surfaces, particularly at the edges of two surfaces.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1a is a longitudinal cross-section of an embodiment of a guide tube device.

FIG. 1b illustrates the insertion of an embodiment of a guide tube device into the opening of the body lumen and the initial deployment process.

FIG. 1c-1e are transverse cross-sections of embodiments of a guide tube device that illustrate the attachment of the spool to the deployment chamber.

FIG. 2a illustrates an embodiment of an endoscope inserted into the deployment nozzle that is attached to the proximal end of deployed guide tube.

FIGS. 2b and 2c illustrate various embodiments of a deployment nozzle.

FIGS. 3a and 3b illustrate an embodiment of the insertion of a deployment nozzle into the proximal end of a guide tube.

FIG. 4 illustrates an alternative embodiment of the pressurization chamber in which the deployment spool has been replaced by an alternative low resistance deployment means formed by a folded guide tube.

FIG. 5 illustrates an alternative configuration of the pressure chamber and guide tube where the guide tube is coiled in a figure eight configuration.

FIG. 6 illustrates an embodiment of the guide tube having stacked loops.

FIGS. 7a-7c illustrates an embodiment of the eversion of a guide tube.

FIG. 8 illustrates an embodiment of a rolled guide tube.

FIG. 9 illustrates an embodiment of a sealed guide tube.

FIGS. 10a-10c illustrate various embodiments of the thread orientation of a guide tube.

FIGS. 11a-11c illustrate an embodiment of a guide tube having a valve that opens upon full eversion of the guide tube.

FIG. 11d illustrates a fully everted guide tube deployed within a body lumen.

FIGS. 12a-12d illustrate various valve embodiments.

FIGS. 13a-13c illustrate additional valve embodiments.

FIGS. 14a-14c illustrate an embodiment a feature that restricts full deployment of the guide tube.

FIGS. 15a-15c illustrate an embodiment of a guide tube with a tether.

FIG. 16 illustrates another embodiment of a tether.

FIG. 17 illustrates the insertion of a semi-rigid tube within a guide tube.

### DETAILED DESCRIPTION OF THE INVENTION

For purposes of clarification, in this document the following definitions will apply:

The terms endoscope or colonoscope refer to a tubular device used for imaging the interior of a body lumen wherein the tubular device is inserted into the body lumen to the site of interest and an image is created for an operator using such means as a fiber optic imaging bundle or electronically such as can be achieved with a camera located on the distal end of the tubular device. The terms endoscopy and colonoscopy will refer to the procedure of using such a device.

When discussing devices such as an endoscope or guide tube, the proximal end will refer to the end to reside outside the body and closest to the operator while the distal end will refer to the end that is to be located furthest from the operator. Furthermore, the terms distal and proximal shall be applied to the guide tube, as if the guide tube is fully deployed (everted). When discussing a body lumen, the proximal end will refer to the end that is deepest within the body while the distal end will refer to that end closest to the outer orifice. In general, the most distal end of the endoscope will be located at the most proximal region of the body lumen.

The terms eversion, evert, everting or other derivations shall refer to the method of deploying a tube within a body cavity. For example, one method of eversion includes applying pressure to an inverted hollow tube to cause it to unfold at its distal end thereby reversing the inverted surface and extending the tube within a body cavity. The tube can be inverted for this purpose by folding the proximal end of the tube back over itself and securing it to the deployment system. Pressure is then applied to the interior cavity created within the tube so that the inverted portion moves through the proximal opening and extends outward, continually unfolding at its endpoint where it is folded back within itself. In one embodiment, the inverted portion of the tube within the interior cavity is flexible such that it is compressed by the applied deployment pressure to cause eversion so that the lumen of the inverted tube is caused to have a volume substantially less than it would in an open, uncompressed state. As the tube is being deployed the entire length of tube will be turned inside out by folding back onto itself. Other means can also be used to create the initial everted tube such as, for example, attaching to a distal end of the tube and pulling part of the tube back within itself so as to turn it partially inside-out. References here to eversion and its derivation are intended to cover these and similar means for partially inverting a tube and the deploying it through the application of internal pressure.

With this invention, we have overcome the limitations of the prior art by reducing the pressure and fluid volume that are required to deploy the guide tube. Furthermore, the invention is less complex than most past systems, thereby making it easier to use and more cost effective to manufacture. The reduction in required pressure is accomplished through incorporation of one or both improvements described within. The first being a low-resistance deployment device and the second, a distal valve incorporated in the guide tube. An example of a low-resistance deployment device is a spool mechanism that allows the guide tube to feed out smoothly and consistently during deployment with little or no resistance while limiting the tube's ability to twist or become entangled. The scheme of spooling the guide tube also limits the amount of air that can enter within the everted tube which is desirable since this air increases the cross section area within the center of the inverted tube and creates resistive drag. Other low-resistance deployment means disclosed here offer these same benefits. An example of a distal valve is also disclosed here. This valve is accomplished by flattening the distal end of the guide tube to limit outward flow of inflation fluid during deployment. This scheme is effective and easier to manufacture than the prior art. These and other features disclosed here provide a simple to use device that is operated with lower pressure and fluid volumes than were previously required, thereby making them safer to use within body cavities.

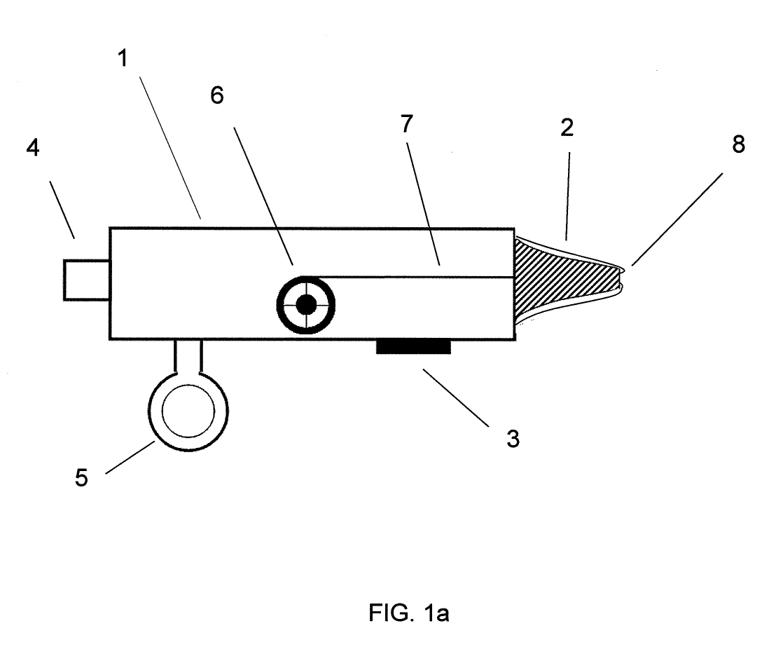

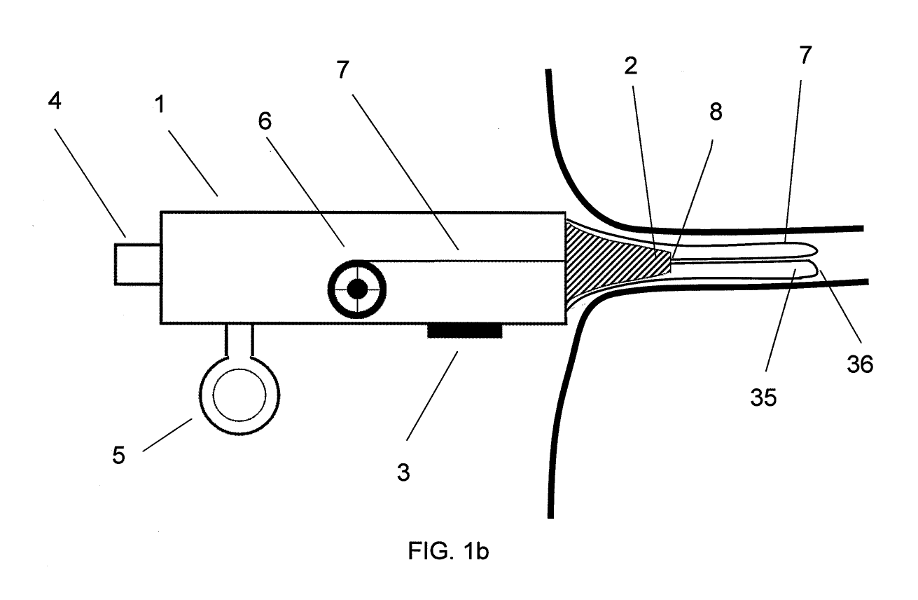

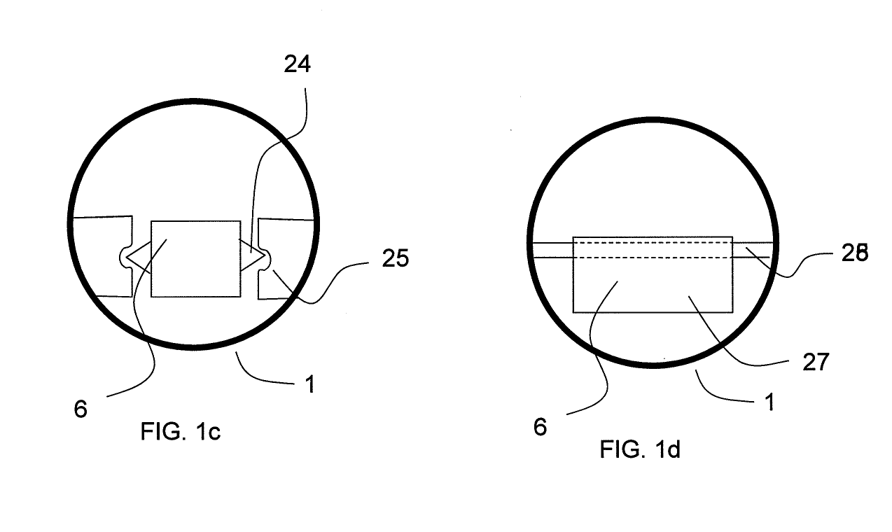

Shown in FIG. 1a, one embodiment of this invention discloses a low-cost effective device that delivers a flexible guide tube 7 within an internal body lumen such as, for example, any tubular internal body lumen, colon, esophagus or an artificially created body tunnel such as is done for NOTES (Natural Orifice Transluminal Endoscopic Surgery). The cylindrical guide tube 7 when placed within an internal body lumen may be used to facilitate the passage of surgical or diagnostic tools within the body lumen. For purposes of illustration the guide tube 7 is drawn here with a cylindrical shape but it is noted that the invention is not limited to any particular cross sectional shape of the guide tube 7 lumen. For example, the cross sectional shape of the guide tube 7 lumen can be substantially rounded, oval, circular or oblong.

In some embodiments, the entire length of the guide tube 7 is deployed by eversion. The deployment of the guide tube 7 can be achieved by the pressure provided by the deployment fluid in communication with the pressure chamber 1. The invention avoids several of the drawbacks of the prior art as exemplified above. This is achieved by employing one or more of the following elements: a single wall tube that is deployed within the body lumen by eversion, an integral valve at the distal end of the tube, a pressurization chamber external to the body to facilitate the eversion deployment process and a means within the pressurization chamber to reduce the friction and resistance that has hindered deployment in previous designs.

FIG. 1b illustrates the pressurization chamber after the deployment nozzle 2 has been inserted into the opening of the body lumen and initial pressure has been applied to initiate the deployment process. The guide tube 7 is depicted in an intermediate state of eversion with some length of the guide tube deployed. There is common communication between the pressurization chamber 1 and the annular cavity 35 of the guide tube. The differential pressure between the pressurization chamber 1 and the interior of the body lumen produces a net outward force at the distal surface 36 of the everting guide tube 7 that results in a forward force to cause eversion and deployment of the guide tube 7 within the body lumen.

The pressurization chamber 1 has several items contained within it and formed on its body. In some embodiments, the pressurization chamber 1 is formed such that its axis is aligned with the direction of the guide tube 7. As a specific example for purposes of explanation; the pressurization chamber is described here as having a cylindrical cross section but it can be other elongated shapes as well, such as one having a rectangular cross section. However, it is preferable that it have a generally elongated shape aligned with the axis of the guide tube 7 and being of a cross-sectional size that can be easily gripped in one hand. When the guide tube 7 is being deployed, the axis of the cylindrical tube of the pressurization chamber 1 is parallel to the axis of the body lumen into which the guide tube 7 is to be deployed. For example, in the case of colonoscopy, the patient lays on their side with their knees pulled toward the chest to allow easier access to the rectum. A right handed physician would then typically grip the pressurization chamber 1 in their left hand and insert the deployment nozzle 2 into the rectum and then apply pressure for deployment with the right hand using either a manual or automated pressurization device. As pressure is applied, force on the guide tube's 7 distal surface begins to cause the guide tube 7 to deploy within the body by means of eversion. The pressurization device could be one of any number of means known to those skilled in the art. Examples of manually pumped pressurization devices are single and double action pumps similar to those used routinely for inflation. Actions for such manual pumps are well known to those in the art and include a piston that is pulled in and out to apply pressure through a valve as well as trigger or lever action schemes similar to those used in caulking and grease guns. As an alternative to pressurization created by manual pumping, the pressurization chamber 1 can be pressurized via a connection to a higher pressure container via inflation port 4. In this case, inflation pressure can be applied either through a valve incorporated within inflation port 4 or either upstream or downstream of it. This approach provides for easier operation since pressure can be delivered via a finger or hand operated switch. In fact, when the action to control the pressurization valve is conveniently located, such as on the body of pressurization chamber 1, this scheme can allow the device to be held in place and pressurized with one hand while leaving the other hand free to perform other tasks.

As the guide tube 7 deploys further into the body lumen, the guide tube's 7 distal end will exit the deployment nozzle 2 and be pulled first into the lumen of the guide tube 7 at its proximal end. As the guide tube 7 is further deployed, the everting guide tube 7 will reach its maximum length at which point the valve at its distal end will open to complete the deployment process. When fully deployed, the guide tube 7 will include a single wall tube that lies within the body lumen with its proximal end outside the body lumen orifice. Full deployment can be detected by a drop in resistance to applied pressure or a drop in pressure in the pressurization chamber either as indicated by the pressure sensor 5 or by feel if using a hand pump. This occurs because the distal end of the guide tube 7 generally remains closed or sealed until eversion is completed, thereby maintaining the pressure in the pressurization chamber 1 as the guide tube 7 is everted. However, once the distal end is opened or unsealed at the completion of eversion, the pressure and pressurization fluid is allowed to dissipate into the body lumen, thereby decreasing the resistance to applied pressure and/or decreasing the pressure in the pressurization chamber 1.

In addition to the deployment nozzle 2, the pressurization chamber 1 has an inflation port 4 as well as an optional a pressure relief valve 3 and an optional port for attachment of a pressure gauge 5. In some embodiments, the pressure relief valve 3 is a simple mechanical device as is well known in the art, an example of which is a hole in the body of the pressurization chamber 1 that is covered by a flexible diaphragm. When the applied pressure within the pressurization chamber 1 exceeds safe limits, the diaphragm will flex to release pressure such that the unit prevents over-pressurization in the event that the operator inadvertently exceeds safe limits. Other valve means can be used to provide a fail-safe mechanism to prevent over-pressurization; the key point here is that one can be included on the body of the pressurization chamber 1 or similarly located so as to be in communication with the pressurization chamber 1.

In addition to the optional pressure relief valve 3, there is an inflation port 4 incorporated into the pressurization chamber 1 so that pressure can be increased within the pressurization chamber 1 so as to cause deployment of the guide tube 7 by eversion. In some embodiments, the fluid used in this pressurization process can be a gas such as air, a fluid means such as water or saline or similar gases or fluids that are well known in the art. In addition to these elements, there can also be a port for attachment of a pressure gauge 5 such that the operator can actively monitor the applied pressure if desired. The device may be made out of a variety of materials such as plastics or metal generally suitable for relatively low operating pressures. For example, in the colonoscopy application, since the colon can burst at pressures of 5 psi, the device can be built from materials suitable for that pressure range. For other applications the device can be built to provide the appropriate pressure, either higher or lower.

In addition to these elements on the body of the pressurization chamber 1, there are also internal elements as shown in FIG. 1b. When using a spool to provide the low resistance means, the guide tube 7 is wound upon and deployed from deployment spool 6. The proximal end of the guide tube 7 coming off the spool 6 is passed through hole 8 located at the distal end of the nozzle 2, everted by folding the tube end back on itself, with the proximal end of the guide tube being attached in a permanent or temporary manner to the inner, outer, or leading surface of the deployment nozzle 2 or other surface of the pressure chamber, forming an air tight seal so that there is little or no fluid leaks from the proximal end of the guide tube 7 during deployment. The end of the guide tube 7 secured to the deployment nozzle 2 forms the proximal end of the guide tube lumen that is being created within the body. The opposite distal end of the guide tube 7 is wound around the deployment spool 6 such that when pressure is applied to the interior space of the pressure chamber 1 via the inflation port 4, the pressure will force the guide tube 7 out of the pressurization chamber 1 through the nozzle 2, causing the deployment spool 6 to rotate as the guide tube 7 deploys. To maximize or increase efficiency of deployment and maintain a relatively low or the lowest possible deployment pressure, the deployment system is a closed system with the exception of the inflation port 4 for fluid input and the pressure relief valve 3. Under normal use, the only expanding point of the system will be the advancing distal surface 36 of the deploying guide tube 7. By design there is no fluid escaping the system under normal operating pressure until the guide tube 7 is fully deployed within the body lumen and distal end of the tube 7 everts, opening the valve at the end of the tube. If the pressure relief valve is incorporated, it will provide a means for an escape of fluid in abnormal over pressurization of the system.

To minimize or reduce the potential for trauma to the body lumen, it is desirable to minimize or reduce the deployment pressure. We have found that the deployment spool 6 provides low resistance such that the guide tube 7 is deployed with such minimal, reduced or low pressure. One means that offers a particularly low resistance is when the outer diameter surface of the deployment spool 6 is generally aligned with the through hole 8 as shown in FIG. 1. This design is preferred when there are no constraints on the diameter of the pressurization chamber 1. For those cases where the diameter of the pressurization chamber is constrained, such as to accommodate the grip of a smaller hand, the center axis of the spool can be moved closer to the center of the pressurization chamber. In some embodiments, the width of the spool 6 is greater than the width of the flattened guide tube 7. For an offset spool such as that shown in FIG. 1, it is desirable that the diameter of the spool is approximately equal to one half the diameter of the pressurization chamber. For a non-offset spool where the center axis of the spool is essentially coincident with the center axis of the pressurization chamber, it is desirable to have the diameter of the spool be slightly less than the diameter of the pressurization chamber. In essence, the diameter of the spool should be as large as possible subject to the constraints of the pressurization chamber and the constraints of the offset axis when the offset axis scheme is used. There are numerous means to configure a spool 6 as is known to those in skilled in the art. For example, to minimize or reduce rotational resistance and complexity one of three methods can be utilized. The first embodiment is where the deployment spool 6 and axle are integral, rotating in unison during deployment as shown in the end view FIG. 1c of the pressure chamber 1. This configuration may be enhanced by the ends of the spool axle terminating in a generally conical shape 24 and mating with a concave depression 25 of the pressure chamber 1 to secure the spool 6. The center rotational points at each end (along the axial direction of the spool) of the deployment spool 6 are longer than the body of the spool 6, providing unhindered rotation of the spool 6. The second embodiment of a spool 6 is shown in the end view FIG. 1d of the spool 6 and axle 28. The spool 6 is a length of cylindrical tube 27 where the majority of the cross sectional area of the cylinder 27 is hollow. The internal diameter of the cylinder 27 is greater than the diameter of the mating axle 28. The axle 28 is a continuous length, passing through the cylinder 27. Its ends are secured within the pressure chamber 1 as shown. As the guide tube is deployed, the cylinder 27 rotates upon the fixed axle 28. The third method (not shown here) is one similar to FIG. 1d in which the axle 28 is offset from the center of the pressurization chamber such that it is located at the center of the spool 6. In this case the spool 6 has spokes connecting the outer rim to a central tube that contains the axle 28.

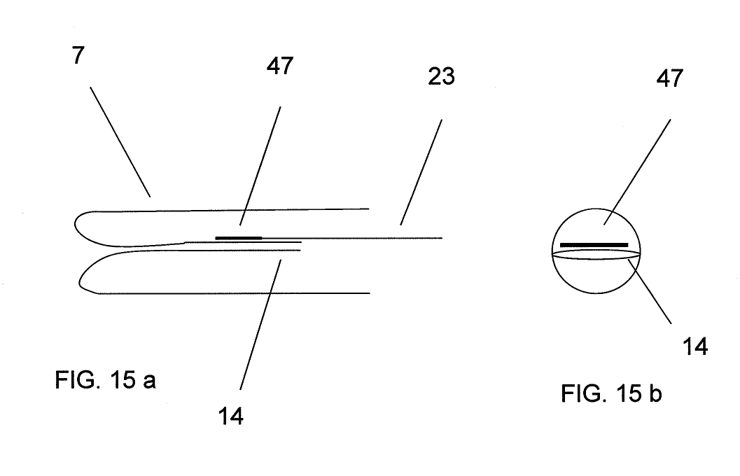

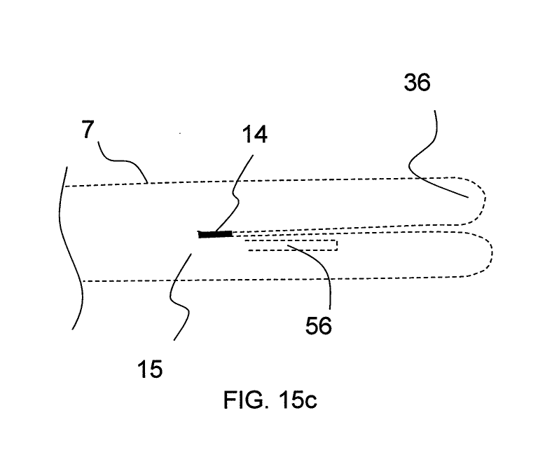

In an alternative configuration shown in FIG. 1e, the deployment spool 6 is fixed to the axel 28 and one of its ends passes through the wall of the pressure chamber 1. A fluid tight seal exists between the axel 28 and pressure chamber 1 wall which the axel 28 passes through. There are various types of bushings or other seals to promote rotation of the axel 28 while maintaining a seal to prevent leakage of fluid when the pressure chamber 1 is internally pressurized. The bushing or other method is used to keep the axel 28 from axial movement within the wall of the pressure chamber 1. The axel end outside the pressure chamber 1 terminates with a wheel 55 that is actuated to cause rotation of the axel in either direction by manual or by motorized means. The forced axel rotation facilitates either deployment or inversion of the guide tube 7. In some instances it is desirable to mechanically control the rate of deployment of the guide tube 7 as can be done by controlling the rate of rotation of the wheel. In some instances it is desirable to be able to cause inversion of the guide tube 7, for instance when pressurization of the deployed guide tube 7 is desired after the initial deployment. In this case the tether 23 described in FIG. 15 would remain attached to the spool 6 so that the distal end of the earlier deployed guide tube 7 may be inverted to cause a seal at its distal end and the guide tube 7 may then be internally pressurized.

Various means are provided to separate the deployed guide tube 7 from the pressure chamber 1 after the guide tube 7 is fully deployed. In one configuration the deployment nozzle 2 can be detached from the pressure chamber 1. A sealing member, such as an O-ring provides a seal between the mating surface of the pressure chamber 1 and the deployment nozzle 2. The union of the two parts can be made by mating threads, locking pins, external collar, clamp, etc. Separating the mating parts requires minimal activation such as a slight opposing twist.

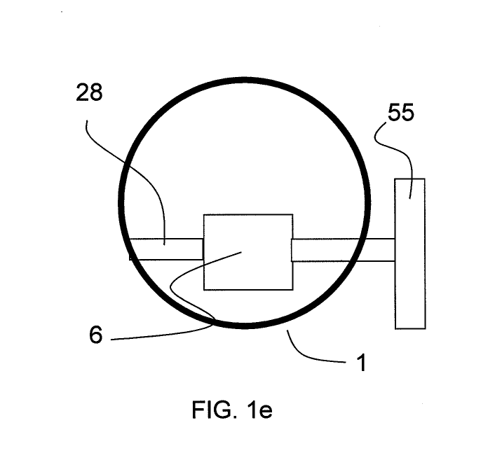

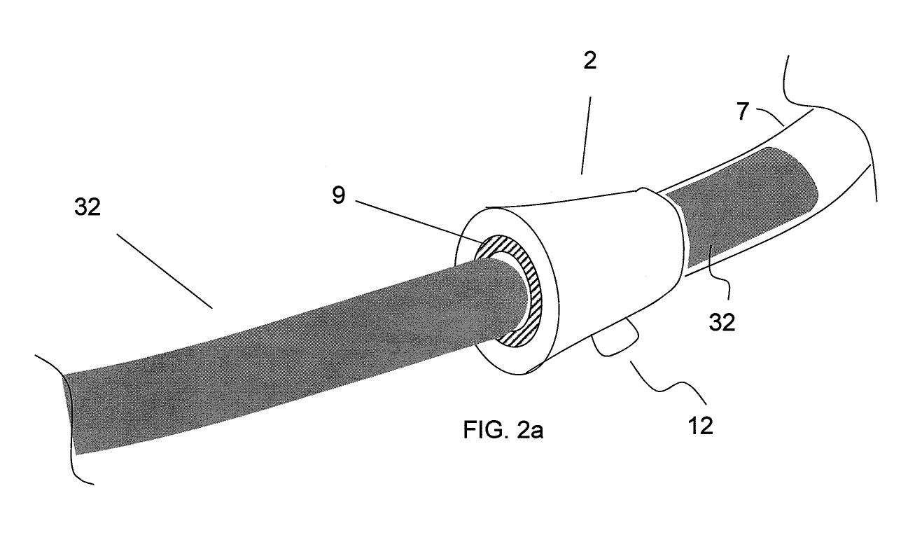

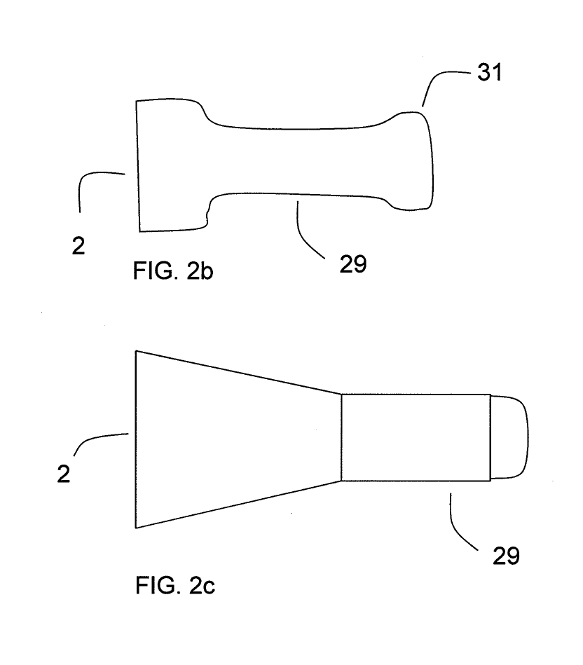

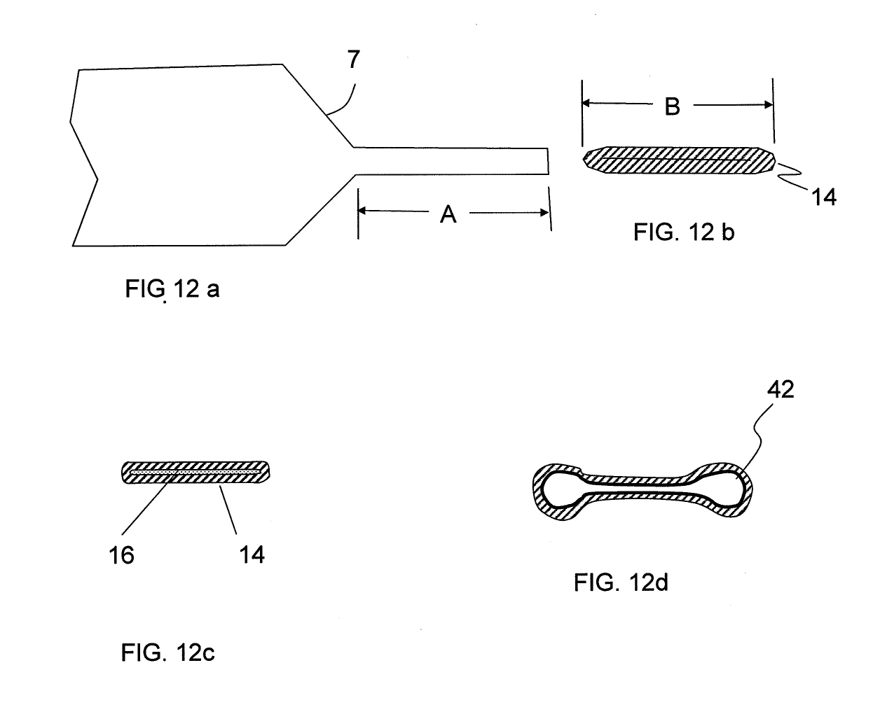

FIG. 2a shows an endoscope 32 inserted into the deployment nozzle 2 that is attached to the proximal end of deployed guide tube 7. In one embodiment, the distal end of the deployed guide tube 7 is temporarily closed. Means for temporarily closing the distal end are discussed in association with FIG. 12. In some embodiments, the deployment nozzle 2 contains an elastomeric sealing grommet 9 having a through hole that is sized slightly smaller than the diameter of the endoscope shaft, in order to ensure a fluid tight seal between the sealing grommet 9 and endoscope shaft. The grommet 9 seals the internal area of the nozzle 2 to the outer surface of the endoscope 32, preventing fluid from escaping from the proximal end of the guide tube 7. The pressure seal at the grommet 9 is maintained as the endoscope 32 is advanced or withdrawn. With a fluid tight seal between the proximal end of the guide tube 7 and the outside surface of the endoscope 32, fluid may be delivered via the endoscope 32 to pressurize the deployed guide tube 7. Alternatively to using an endoscope 32 to pressurize the guide tube 7, an alternative embodiment incorporates a port 12 in the wall of the deployment nozzle 2 and distal to the grommet 9 in order to pressurize the guide tube 7 from an alternative pressure source. The grommet 9 may be either cut from an elastomer sheet or molded.

As shown in FIG. 2b-2c, the distal end of the deployment nozzle 2 may have various shapes. FIG. 2b shows one embodiment with an extended length distal cylinder 29 whose distal end 31 has a larger diameter than the more proximal shaft diameter section 29. The distal end 31 may also be comprised of a softer material such as an elastomer, so as to be flexible.

In another embodiment of the deployment nozzle 2, illustrated in FIG. 2c, the nozzle 2 and components comprising the nozzle 2 may be separated into 2 or more pieces each, to laterally disengage the nozzle 2 from the endoscope 32. As an example, in this configuration the nozzle 2 is formed as two mating halves that snap together around the central lumen. For removal from the endoscope these two pieces are unsnapped with one being moved away from the endoscope 32 to the left and the other being removed away from the endoscope 32 to the right.

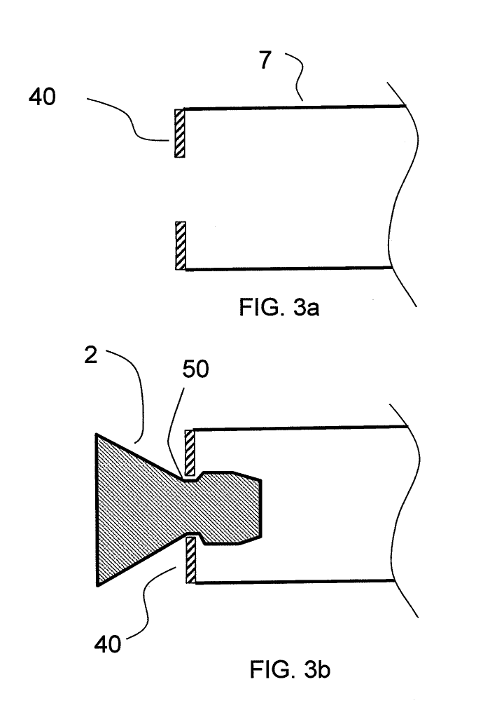

In FIG. 3a the proximal end of the guide tube 7 has an elastomeric ring 40 with an outside surface diameter and a through hole. The ring's outer diameter is adhered to the proximal end of the guide tube 7. Its through hole is sized slightly smaller than the mating diameter of the deployment nozzle 2 and the endoscope 32. The elastomeric ring 40 provides a fluid tight seal between the proximal end of the guide tube 7 and the mating surface of the deployment nozzle 2 and endoscope 32. As shown in FIG. 3b, the deployment nozzle 2 surface can be configured with an annular indent 50 that is perpendicular to its axis. When the nozzle 2 is inserted into the elastomeric ring 40, the elastomeric ring 40 seats within this indented section of the deployment nozzle 2 as shown in FIG. 3b. The through hole of the elastomeric ring 40 can be sized slightly smaller than the diameter of the annular indent 50, and the distal portion of the deployment nozzle 2 can be greater in diameter than the annular indent 50, which helps the elastomeric ring 40 retain the deployment nozzle 2 in place after insertion. During deployment of the guide tube 7 the elastomeric ring 40 is secured around the deployment nozzle as shown in FIG. 3b. In some embodiments, prior to guide tube 7 deployment, the proximal end the guide tube is passed through the nozzle in the proximal to distal direction and everted by rolling the proximal end of the guide tube over the distal end of the nozzle where the elastomeric ring 40 seats to the annular indent 50. Once guide tube 7 deployment is achieved, its proximal end with the elastomeric ring 40 may be removed from the deployment nozzle 2 and an endoscope 32 inserted. In some embodiments, the elastomeric ring 40 can be formed by die cutting from an elastomeric sheet or it can be molded. In another embodiment where a seal between the endoscope shaft diameter and the elastomeric ring's 40 through hole is not desired, the elastomeric ring's 40 through hole is sized larger than the mating endoscope shaft diameter but not larger than the diameter of the annular indent 50. There are numerous other methods to secure the proximal end of the guide tube 7 to the deployment nozzle 2 as know by those skilled in the arts, including using a clamp, collar, elastomeric or rigid retention ring, tie, interference fit of mating surfaces, etc.

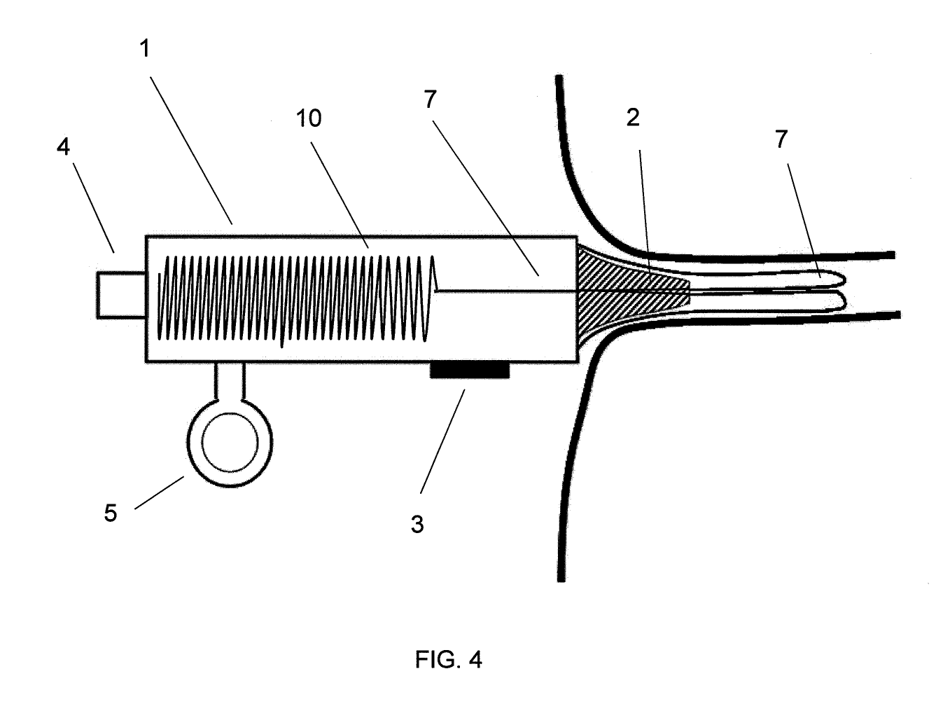

FIG. 4 illustrates an alternative embodiment of the pressurization chamber 1 in which the deployment spool has been replaced by an alternative low resistance deployment means formed by a folded guide tube 10 in which the guide tube is folded in the manner shown. The guide tube 7 is folded into a bundle 10 such that, as the guide tube 7 is deployed, the direction of deployment is orthogonal to the folds. The significance of the concept disclosed here is that it minimizes or reduces the resistance as the guide tube 7 is deployed such that the guide tube 7 can be deployed at a reduced, low, or lowest pressure possible. Alternative folding schemes described in the prior art can be inferior to this approach since they generally require the deploying guide tube to be pulled along itself as each fold deploys which significantly adds to the deployment resistance. The technique disclosed herein, with the orthogonal folding scheme eliminates or reduces this resistive component.

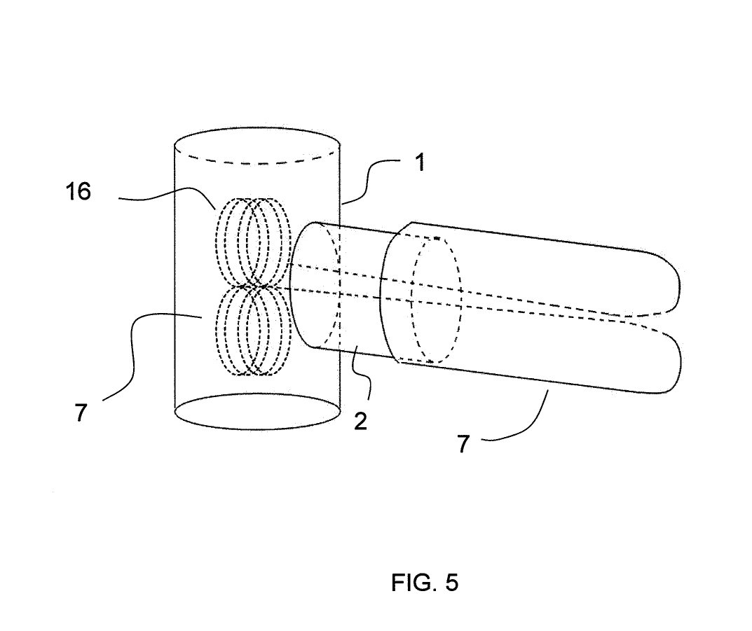

FIG. 5 illustrates an alternative configuration of the pressure chamber 1 and guide tube 7 prior to guide tube 7 deployment. In this configuration the guide tube 7 is coiled in a figure eight 16, either orthogonally or parallel to the direction of deployment and nozzle 2, the central axis of the pressure chamber 1 can be configured to be orthogonal to axis of the guide tube 7 as shown in the figure or it can be oriented parallel to it as shown in the design in FIG. 1. As the guide tube 7 deploys, it will not suffer significant kinks or twists as a result of dispensing from the figure eight configuration. The figure eight configuration is made by placing the guide tube 7 in two opposing loops, alternating the winding orientation between one loop and then the other. Each respective loop is wound in the same direction, but wound in a direction opposite of the other. For example, the top loop of the figure eight is wound in the clockwise direction for one revolution, followed by a complete revolution in the counter-clockwise direction forming the bottom loop. This sequence is repeated for the entire length of guide tube 7. Each loop places a slight twist in that length of guide tube 7. With the opposing loops being wound in opposite directions, the axial twist of a length resulting from a loop made in the clockwise direction will have a twist orientation opposite of the opposing loop made in the counter-clockwise direction. Consequently, any twisting that results from one half of the figure eight loop is countered by the opposite twist that occurs when the opposing figure eight loop deploys. This scheme also provides a low resistance method of deploying the guide tube 7, thereby resulting in a deployment pressure that is lower than generally used in the prior art.

An alternative to the figure eight coiling configuration is to stack individual loops of the guide tube 7 on top of the next as shown in the isometric view of FIG. 6. This coil configuration is achieved by stacking each coil on the next without causing the end of the tube to twist as shown in FIG. 6. For example, this guide tube 7 configuration can be created by gripping the guide tube 7 from underneath using right hand, making a loop of the section of tube by rotating the hand 180 degrees in the counter clockwise direction and then grasping the adjacent length in same manner and repeating. As each coil unwinds the coil will straighten and the 180 degree rotation used to create the loop will reverse and there will be no residual twist in the deployed guide tube 7. This also results in a low resistance deployment scheme that requires minimal deployment pressure.

The guide tube 7 is typically formed from a long section of tubing as is known in the art. An example of a material for this guide tube 7 is low durometer polyurethane. In some embodiments, the guide tube 7 is formed by a seamless process such as blow molding of an extruded tube to achieve the desired tube diameter and wall thickness. Blow molding of the guide tube 7 as compared to a guide tube that is extruded to final dimensions, provides superior strength and better resistance to structural deformity when the resulting tube is pressurized during intended use. The diameter of the guide tube 7 is sized to be larger than the diameter of the endoscope 32 but not generally larger than the internal diameter of the body lumen. The guide tube 7 wall thickness is to be held as thin as possible to promote eversion yet thick enough to provide an adequate safety factor such that the guide tube 7 does not distend from internal pressure or become damaged from endoscope 32 passage. Minimum wall thickness of the guide tube 7 must be adequate to accommodate the hoop stress from pressurization. For example, in some embodiments, for a 14 mm diameter tube a wall thickness or 0.005″-0.010″ is functional. However, the wall thickness may be less or greater. The wall of the tube may be laminated, and include one or more materials or similar materials with varying specific properties, such as durometer, elasticity, Young's modulus, and other properties. In one embodiment, an example of optimal material property characteristics for a 14 mm diameter guide tube 7 would be a wall thickness equal to or less than 0.005″. Material that is very flexible as to promote eversion without adding resistance, does not distend when pressurized up to 10 psi and for those situations where the physician desires to view the body luminal surface as the endoscope is advanced, a transparent tube is preferred in some embodiments.

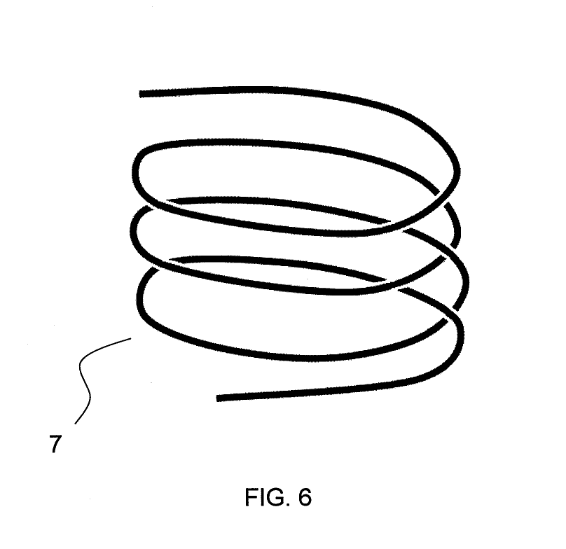

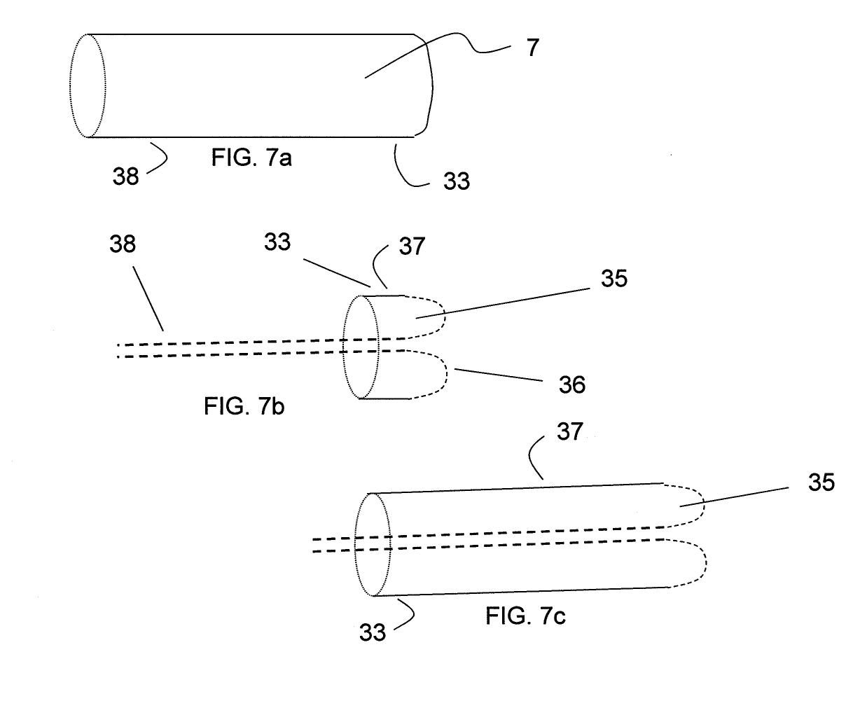

As shown in FIG. 7a, the guide tube 7 is a single wall tube, wherein the proximal end 33 is everted by rolling back over, onto itself as shown in FIG. 7b. This creates a small annular cavity 35 wherein pressure is applied to evert the tube 7. The balance of the tube 7 is compressed by the pressure within the annular cavity 35. As pressure is applied and the guide tube 7 begins to evert, the length of the annular cavity 35 increases as shown in FIG. 7b and FIG. 7c. Note that during eversion this cavity 35 will have three surfaces. The first is the outer surface 37 formed by the outer wall of the everted guide tube 7. The second is the inner surface 38 that is formed by the section of non-everted guide tube 7. Ideally this inner surface 38 is of small diameter and it passes right down the center of the everted guide tube 7. We will refer to these two surfaces as the outer surface 37 and the inner surface 38 of the guide tube 7. The third surface, which we will refer to as the distal surface or distal fold 36, is formed at the end of the tube 7 where the tube 7 is everting or folding back upon itself, effectively connecting the outer surface 37 to the inner surface 38. Pressure for eversion is applied to this cavity 35 at the proximal end 33 of the guide tube 7 where communication to the pressurization chamber 1 exists due to the fact that the inner and outer surfaces are not connected to each other. When fully deployed the cavity 35 extends along the full length of the guide tube 7, i.e. when fully deployed the volume of this internal cavity 35 is simply the volume of the everted guide tube 7.

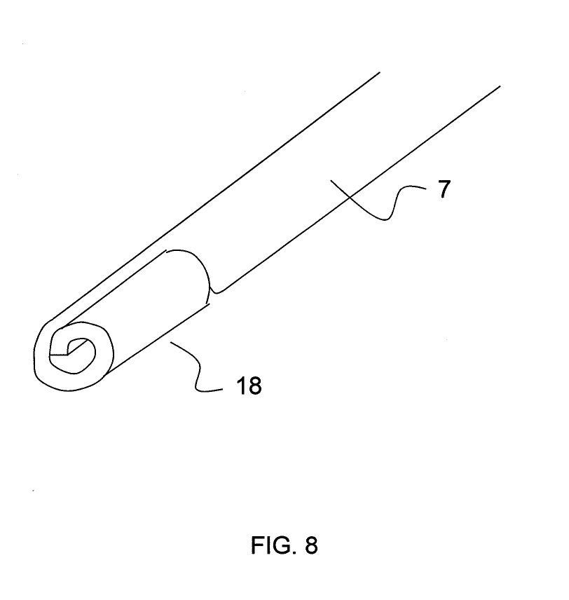

In FIG. 8 an alternate means is described to minimize the cross section of the inner surface where the guide tube 7 is rolled along the longitudinal axis its entire length. Many thermal plastics will maintain a memory or what is often referred to as “set” when configured in a rolled orientation for an extended period of time, or a heat process may be used to partially set the guide tube 7 in this configuration. The roll 18 minimizes the contact surface area between the overlapping inner and outer guide tube during deployment and therefore reduces the resistive forces detrimental to deployment. The tube 7 releases from the rolled configuration at the distal surface during eversion, as the non-everted guide tube 7 transitions to become the outer guide tube.

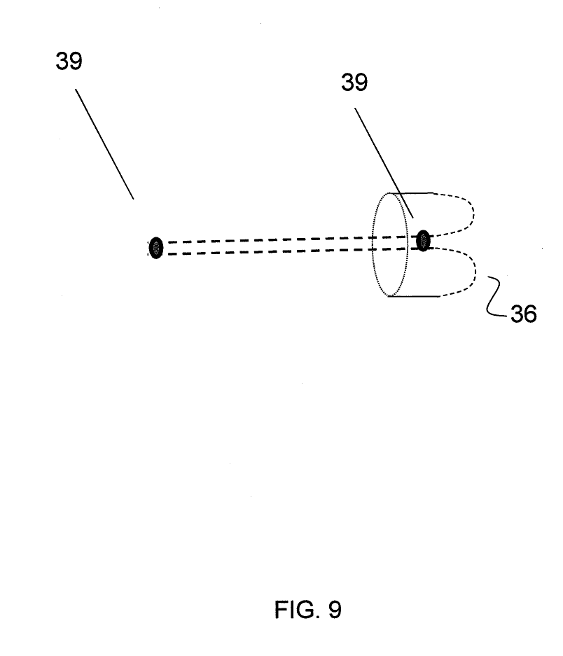

In the embodiments shown in FIG. 7 or that shown in FIG. 8, for example, an additional step may be employed wherein seals 39A, 39B are formed at both ends of the inner guide tube as illustrated in FIG. 9. After sealing, a vacuum pressure is applied to the cavity formed between the seals 39A, 39B such that the volume that exists within the inner tube surface is minimized or reduced, thereby reducing the total surface area of the inner tube. Both seals 39A, 39B are temporary. Once the distal surface 36 of the guide tube 7 is inserted into the body lumen and fluid pressure is applied within the annular space 35, seals 39A, 39B can be broken since the applied fluid pressure on the inner guide tube will exert a sealing pressure on the inner guide tube that will prevent subsequent air pockets from forming. As outlined below, these seals 39A, 39B can be formed in a number of ways such as by applying temporary pressure while the vacuum is pulled and then rolling or folding the guide tube, applying an adhesive seal, such as an epoxy seal, that breaks when eversion pressure begins to deploy the tube or thermal sealing to create a flattened section of the tube.

When such a temporary vacuum seal is applied to the inner volume, the seal can be formed at the time when the device is manufactured and packaged or it can formed by the user just prior to guide tube 7 deployment. When the vacuum is created by the user, a syringe or similar device is attached to the distal end of the nozzle 2 and a vacuum is created by withdrawing the plunger of the syringe, after which pressure is applied to the pressurization chamber 1 to help prevent fluid from reentering the inverted guide tube. Alternatively, the order of these two operations can be reversed.

Note that such a vacuum and pressurization process effectively self-seals the tube, i.e. no discrete additional sealing steps need be applied at the locations designated 39A, 39B. In such an embodiment, this vacuum is held in place by applying a temporary seal to prevent air or fluid from reentering the inverted guide tube. When the vacuum is applied at the time of manufacture a temporary seal is created around the inner surface of the guide tube to maintain the vacuum prior to tube deployment. Alternatively, the vacuum seal can be a cap placed over the end of the nozzle 2 and removed prior to deployment. The seal can be a temporary seal as is commonly used in packaging processes. In that case the seal breaks when sufficient pressure is applied to the pressurization chamber 1 to begin deployment. Alternatively, the seal can be one that is applied to the end of the nozzle 2 either at the time of manufacture or by the user, and then removed just prior to deployment.

It is important to note that this vacuum sealing does not need to be perfect. While placement of a temporary seal can be used to maintain a higher vacuum, it should be noted that after the air within the inverted guide tube is removed by the vacuum, the guide tube 7 tends to naturally seal upon itself such that it is difficult for air to reenter the inverted tube prior to deployment. This is true for both the configuration shown in FIG. 1 as well as the configuration shown in FIG. 4.

The key element here is incorporation of a means to create a vacuum within the inner guide tube in order to reduce its overall profile which subsequently reduces the frictional forces that are created during deployment.

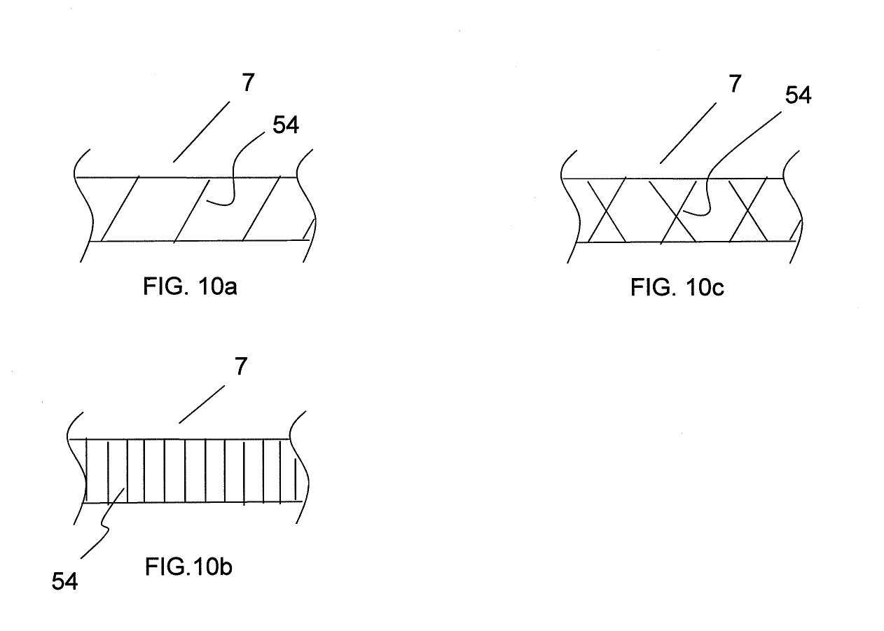

In another embodiment, a modified guide tube 7 is constructed using a flexible ribbed structure 54 constructed by spirally embedding or wrapping a continuous length of material, such as a thread, with a higher elastic modulus, i.e. a greater stiffness, than the guide tube wall material within or on the tube wall with spacing between the sections as shown in FIG. 10a. We will refer to this type of construction as a segmented guide tube 7. When the deployed segmented guide tube 7 is internally pressurized, the higher elastic modulus material restricts expansion along its length while allowing for expansion in the regions in between. In other words, expansion is largest in the middle of the segments created by the higher elastic modulus material but it is negligible or reduced at the edges of these segments.

In a non-segmented guide tube 7, it is the nature of the guide tube 7 to be straight when internally pressurized within a straight body lumen but at a fold, the tube wall at the inside apex of the bend is pressed against the opposing side of the tube, which can partially or completely occlude the lumen of the guide tube. When a segmented guide tube is internally pressurized and placed into a bend, the tube will partially fold within the bend at multiple defined bend points. In essence, the incorporation of the higher tensile strength or a stiffer material as disclosed here provides bend points for multiple small folds to occur within a bend at the incorporated stiffer material thereby reducing the likelihood of a single large fold forming and completely occluding the inner lumen of the tube.

We have found that shorter segment lengths result in a lower likelihood of such a single completely occluding fold occurring. The orientation of the segments is generally perpendicular to the tube axis. As a specific example, thread can be used to form the segmented guide tube. Thread is flexible and does not significantly hinder the deployment properties of the guide tube 7. There are numerous methods for fabricating these controlled bend points within a pressurized guide tube, including laminating one or more threads between tube layers or dip coating a like material layer over a tube with the structural threads in place.

The thread orientation within the tube may be of a variety of configurations. FIG. 10b illustrates the use of discrete circular thread rings as one example. Alternatively, FIG. 10c illustrates segments formed by wrapping the tube along its length first with one thread in a clockwise manner followed by wrapping with another thread in a counter-clockwise manner to form a mesh or weave pattern. Any number of such embodiments can be employed, the key feature being the use of an everting guide tube primarily formed of a highly flexible material and incorporated within it or on it a second material having a higher elastic modulus to form segmented regions along the length of the guide tube.

In another configuration of a guide tube 7 with controlled bend points, rings perpendicular to the axis spaced at a set interval are molded into the tube. In some embodiments, the cross sectional areas enclosed by each ring is greater than the cross section of the tube wall thereby creating ribs along the outer surface of the tube wall. Controlled bend points with a guide tube may also be achieved by molding a tube with defined segments where the segments are separated by a short length where the cross sectional area is either increased or decreased relative to the nominal cross sectional area of the tube.

Frictional forces acting on deployment guide tube 7 arise when the inner and outer surface of the tube pass over each other during deployment. These can be reduced by reducing the coefficient of friction of the guide tube 7 surface. The tube surface can be modified in a variety of ways, including plasma energy treatment, bonding a wetting material to the surfaces such as polyvinylypyrrolidone (PVP), hyaluronic acid, or applying a surface coating such as parylene, etc. Another option to reduce the friction associated with the guide tube 7 is to apply a lubricating agent directly to the guide tube surface prior to deployment. Lubricating agents can be selected from a range of biocompatible lubricants as are well known in the art. In some embodiments, the guide tube surface can be modified to reduce the coefficient of friction by one of the above surface treatment methods and/or use of biocompatible lubricants that are known and acceptable for mucosal contact and are primarily comprised of glycerin, and propylene glycol.

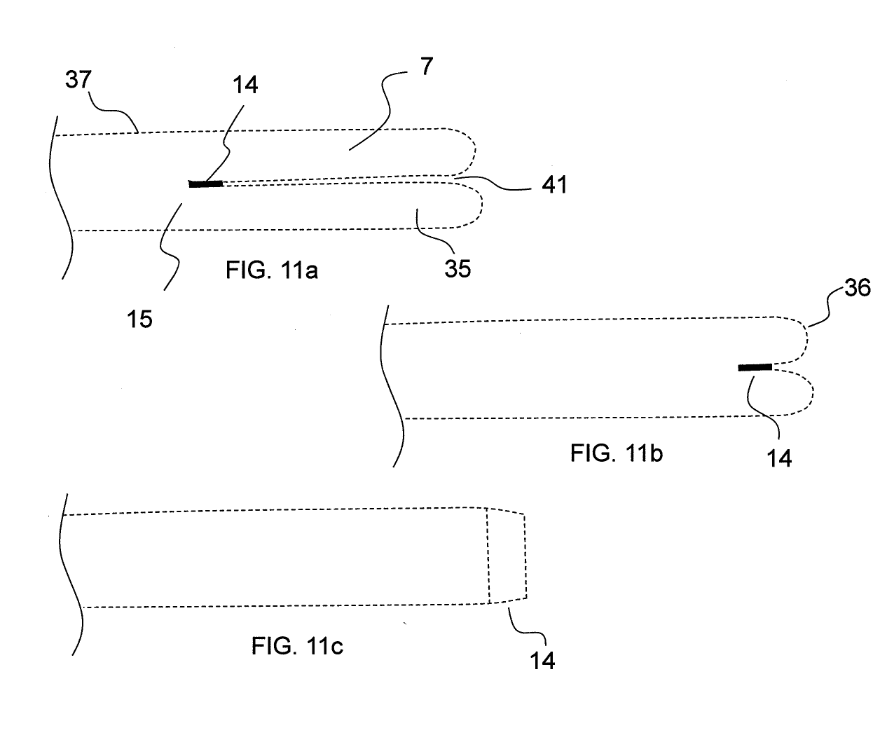

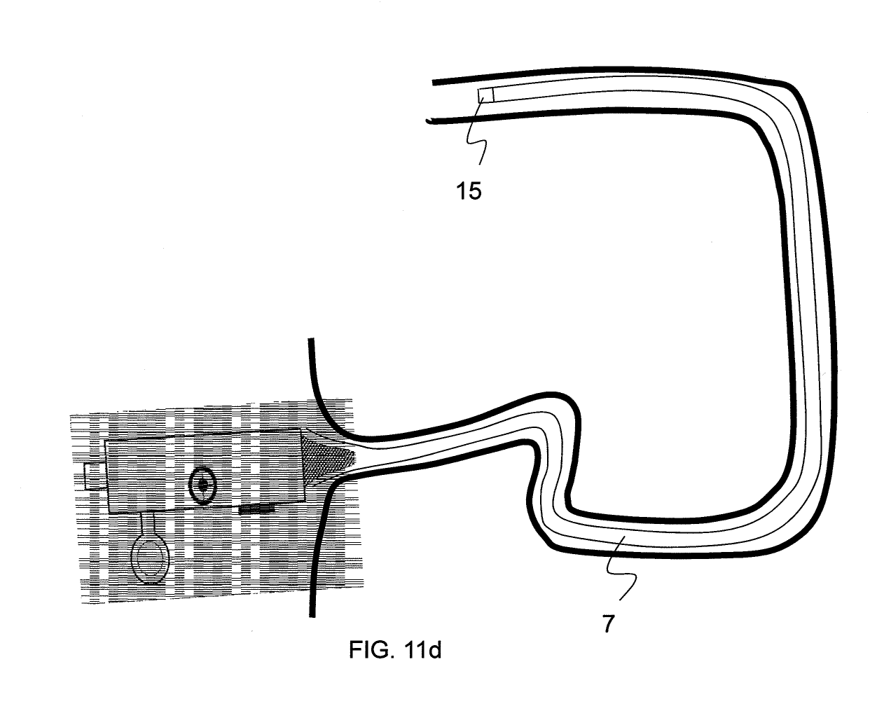

As shown in FIG. 11a, the guide tube 7 incorporates a passive valve 14 (described below) at its distal end, sealing the guide tube 7 to fluid passage during eversion. The valve opens after the section of guide tube 7 in which it resides is everted. The purpose of the valve 14 is to provide a means to perfect the seal of the distal end 15 of the guide tube 7 in order to limit or prevent fluid loss through the lumen of inner surface 41 while the guide tube 7 is everting during deployment. The mechanics of guide tube 7 deployment are discussed in association with FIG. 7. FIG. 11a shows the distal end 15 of the guide tube 7 within the outer surface 37. In some embodiments, prior to one half of the guide tube length being deployed, distal end 15 is located within the pressurization chamber 1 but once approximately one half of the guide tube length is deployed, location 15 is located within the surface 37 as illustrated in FIG. 11a. The interior area of the guide tube 7 is subjected to fluid pressure within the annular cavity 35. The pressure within the annular cavity 35 is higher than the pressure within the body lumen, acting to seal the passive valve 14 and evert the guide tube 7. FIG. 11b illustrates the guide tube 7 deployment just prior to full deployment where the guide tube 7 deployment is nearly complete and the valve 14 is nearing the distal surface 36. When the guide tube 7 eversion progresses to the point the passive valve 14 reaches the distal surface, it will evert and open the distal end 15 of the guide tube 7 to the body as shown in FIG. 11c.

FIG. 11d shows the guide tube 7 fully deployed within a body lumen such as a colon. In the fully deployed state, the guide tube 7 is in a single wall tube configuration along its entire length. The valve 14 at the distal end 15 of the guide tube 7 is everted and open to the body lumen.

FIG. 12a-FIG. 12b depict an embodiment of the passive valve 14. In this embodiment, the passive valve 14 is formed from the body of the guide tube 7 by flattening the distal end 15 of the guide tube 7 as shown in the end view of FIG. 12b. In some embodiments, the valve 14 can have a length A which is approximately equal to the diameter of the tube. In other embodiments, the valve can have a length A which is longer or shorter than the diameter of the tube. When flattened, the valve has a width B. The two surfaces that form the top and bottom of the passive valve 14 lie flat upon the other. Prior to being fully formed in this embodiment, the flattened guide tube 7 has internal radii of curvature 42 on each edge as illustrated in FIG. 12d. Specifically it is noted here that the illustration shown in FIG. 12d is another embodiment since this structure has cavities formed along each edge by the two radii of curvature 42. If left in this non-creased configuration, these internal radii at the outer folds of the tube would create a fluid path when the tube is everting during deployment. The passive valve 14 in some embodiments is illustrated in FIG. 12b and FIG. 12c where the distal end of the tube is altered in a manner to eliminate the two cavities shown in FIG. 12d. With this embodiment, the pressure exerted on the outside of the valve 14 effectively seals the distal end 15 during guide tube 7 deployment. Valves 14 much shorter than the length A described here generally do not seal as well and are therefore not preferred in some embodiments. Valves much longer than length A are not necessarily preferred in some embodiments since they do not significantly enhance the seal and ultimately add to the deployment resistance. In some embodiments, the flattened cross section of passive valve 14 can be achieved by heating a polymer guide tube 7 to the glass transition temperature of the material while pressing the tube end between two flat forming plates, causing the guide tube to flatten into the cross section shown in FIG. 12b. By heating the guide tube end in this manner, the mating surfaces of the tube in the valve seal region remain generally in contact with each other rather than forming the channels at the outer edges of the valve that arise from the two radii of curvature 42 that are present when this heating process is not undertaken. As desired, the flattened tube everts with low resistance when the guide tube 7 has deployed to its final length. Furthermore, to aid the sealing of the valve, the flattened section 14 can be filled with a viscous fluid-like material 16, suitable for internal body use, an example of which is silicone fluid or gel. This is shown in FIG. 12c. The advantages of this and the other valves disclosed below are the reduced fluid volume needed for deployment and the reduction in deployment pressure required.

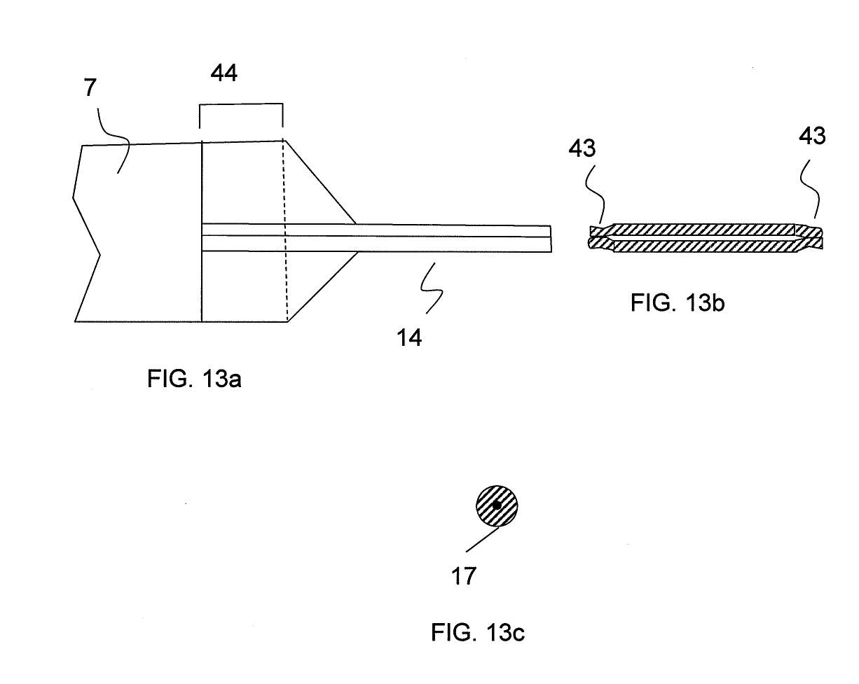

FIG. 13a-FIG. 13c discloses an alternative distal passive valve 14 configuration of the guide tube 7. This valve 14 is shown in side view in FIG. 13a and in end view in FIG. 13b. It is formed by sealing the outside edges 43 of two adjacent flat sheets. In some embodiments, the formed valve assembly has a length greater than its width. In other embodiments, the valve assembly has a length approximately or substantially equal to its width. In yet another embodiment, the valve assembly has a length less than its width. It is bonded onto the end of the guide tube as shown in FIG. 13a. The bond area 44 shows the region where the valve assembly is secured to the tube.

A further embodiment of the distal passive valve 14 is shown in FIG. 13c. Here the valve is formed by an elastic material located at the distal end 15 of the guide tube 7. This elastic valve 17 can be formed by bonding a cylindrical section of an elastic material, such as an elastomer, to the distal end of the guide tube. When un-stretched, the elastic cylinder's nominal internal diameter is essentially or substantially zero such that the unstretched elastic cylinder forms a narrow tube through which there is significant resistance to the passage of the inflation fluid, thereby forming a seal. However, when stretched, the internal diameter of this elastic cylindrical section is sufficiently large to allow the passage of relevant medical instrumentation such as an endoscope.

Alternatively to using a valve 14 to seal the distal end 15 of the guide tube 7 during eversion, viscous fluid such as silicone may be located within the lumen of the inner surface. Referring to FIG. 11a, the addition of viscous fluid within the lumen of the inner surface 41 serves as a seal to minimize or reduce loss of pressurization fluid resulting from fluid passage from the annular cavity 35 into the cavity 41. The viscous fluid may reside within the entire length of the lumen of the inner surface or only at the distal end of the lumen of the inner surface. The pressure of the annular cavity is higher then the pressure in the body lumen and the lumen of the inner surface of the guide tube. That differential pressure acts to minimize, reduce or close the gaps for fluid loss during deployment. In one embodiment, vicious fluid is first applied to the interior of the guide tube 7. Next it is tightly rolled onto the deployment spool 6. When the guide tube 7 is subsequently deployed by eversion the vicious fluid within inner surface 41 helps to seal the lumen from pressure leaks originating in chamber 35. This vicious fluid also helps to minimize or reduce the volume contained within inner surface 41 by minimizing or reducing the likelihood that air can enter this cavity from the distal opening located adjacent to distal fold 36. By preventing or reducing this reentry of air, the cross section of the non-inverted portion of the guide tube is reduced which also minimizes or reduces deployment resistance.

Other means can be used to form a valve or temporary seal at the distal end of the guide tube to promote eversion. Examples of alternative means to accomplish this are folding the distal end of the guide tube, use of a balloon, snare, pressure sensitive adhesive, adhesive, thermal or other applied energy tacking or welding, solvent bonding, elastic or inelastic band, and the like.

Various means have been disclosed separately here for achieving either guide tube deployment with low resistance pressure and for providing a distal valve to reduce the required deployment pressure and the volume of inflation fluid required. We also note that we have determined through bench-top testing that guide tube 7 deployment is further enhanced when these two features are used in combination. Specifically, we have found that, and we disclose here, that a combination of these two features using any one of the low resistance means disclosed here with any of the distal valve or sealing schemes disclosed here or elsewhere in the prior art provides performance superior to those concepts that exist in the prior art.

The function of the guide tube 7 may be enhanced with a distal valve 14 that does not open immediately during the guide tube 7 deployment so that the deployed guide tube 7 can be internally pressurized as the endoscope is being advanced within it. This is accomplished by the guide tube 7 deployment being stopped when the distal valve 14 has not yet reached but is in close proximity of the distal fold 36.

By not everting the last few inches of the guide tube 7 during deployment the distal valve 14 remains sealed. The proximal end of the guide tube 7 is sealed to the shaft of an endoscope by means such as that described by the description associated with FIG. 2a. An endoscope is inserted past the proximal end of guide tube seal and the guide tube lumen is pressurized with fluid delivered from the endoscope. This creates an open space within the guide tube lumen in which the endoscope is advanced to the tube's distal end. To open the distal end 15 of the guide tube 7, the distal seal is mechanically released or opened, providing endoscope access beyond the end of the guide tube 7.

Pressurizing the guide tube 7 during endoscopic advancement provides a number of features to facilitate the insertion of the endoscope within the guide tube 7. The guide tube 7 has improved column strength to better fix the distal end in place as the endoscope is being advanced. In addition the opening in the lumen of the guide tube 7 is generally increased, thereby easing endoscope passage. A pressurized guide tube 7 also has the natural tendency to be in a straightened configuration. Therefore, when the deployed guide tube 7 is pressurized, the straightening effect of the guide tube 7 provides support to minimize or reduce the severity of bends within the colon caused by the advancing endoscope.

In one embodiment full deployment is temporarily stopped by fixing the distal end of the guide tube with a temporary seal known to those skilled in the art. Examples of such temporary seals are application of epoxy or adhesive at the distal end of the guide tube 7, thermally sealing the distal end of guide tube 7 or application of a stricture such as band or o-ring seal to the distal end of guide tube 7. The seals disclosed here are stronger than those described above, i.e. the burst pressure of these seals is generally in the range of 3-5 psi or above such that they will not open under normal deployment pressure. As a consequence, when the guide tube 7 is deploying, deployment stops when the distal seal is reached. Added pressure at this point would be noticeable to the operator and would result in opening of the pressure relief valve 3 if it is employed. When this point is reached the operator can advance the endoscope as described above. When the endoscope reaches the sealed end of the guide tube 7 it can be pushed through the distal end by via pushing force from the operator. Alternatively the operator can open the distal seal using the tools that are available via the distal end of the endoscope.

In yet another embodiment, the distal end of the guide tube 7 is permanently sealed. In this case the device is operated in the same manner with the endoscope inserted until it reaches the seal at the distal end. Once this is accomplished the operator uses a cutting tool in the endoscope to puncture this distal seal and advance the endoscope beyond the distal end of the guide tube 7.

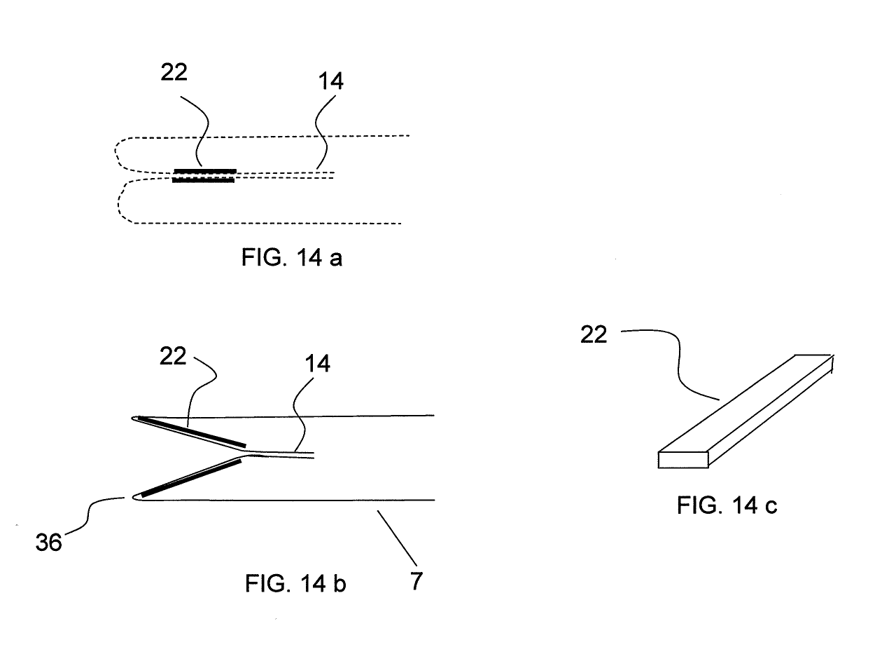

In another embodiment the full deployment is temporarily stopped by mechanical means incorporated into the guide tube 7. An embodiment of a mechanical means to stop the deployment of the guide tube 7 during eversion is shown in FIGS. 14a-c. As illustrated in FIGS. 14a and 14c, one or more structural splines 22 are molded or bonded into a common cross section of guide tube wall, proximal to valve 14. The splines 22 are preferably configured from a plastic that is significantly stiffer than the material used for the guide tube 7. Each spline 22 is oriented in a configuration parallel to the axis of the guide tube 7 and is an integral part of the guide tube wall. The splines 22 are of adequate stiffness and length to temporarily stop the portion of the guide tube where the splines 22 are located from everting during deployment. As the distal surface 36 becomes aligned with the splines, the splines' length prevent them from inverting in the confines of the tube lumen, as illustrated in FIG. 14b. The applied deployment pressure when this point is reached is not increased substantially above the nominal value applied during the initial deployment phase, temporarily stopping the guide tube 7 deployment. This results in valve 14 remaining sealed and the ability of the guide tube 7 to be internally pressurized as an endoscope is advanced through its lumen. To complete the eversion of distal end of the guide tube 7, the endoscope is advanced, pressing into the splines 22 and causing adequate force at the distal surface to evert the guide tube length containing the splines 22.

One configuration disclosed here maintains temporary static pressurization of the guide tube 7 by using a tether 23 to mechanically restrain the distal end of the guide tube 7 to thereby prevent or inhibit the distal end from everting during initial deployment. Means to achieve this function are shown in FIGS. 15a and 15b, which illustrate a side view and a front view of the distal portion of the guide tube 7, respectively. The distal end of the tether 23 is attached to one or more points of a common cross section on the guide tube wall, at a location proximal of the valve 14, or at the valve 14, or distal to the valve 14. In some embodiments, the valve can be flat, although other valve types as described herein are also contemplated. In some embodiments, the tether 23 can be attached to the guide tube wall with wall anchor 47. In some embodiments, the proximal end of the tether 23 has a mechanical stop that restricts the length of the tether 23 that is fed into the guide tube 7 during eversion, thus preventing full deployment of the guide tube 7. When used in conjunction with deployment spool 6 the length of the tether 23 is wrapped onto the spool followed by the guide tube 7. Here the end of the tether 23 terminates with a stop. An example of such a stop is a semi rigid tab having a length and width greater than the through hole of the deployment nozzle 2, thereby preventing the tab from passing through the deployment nozzle 2. During guide tube 7 deployment, when the tab encounters the deployment nozzle 2 the tether 23 draws taught and prevents valve 14 from everting. The guide tube 7 can then be internally pressurized while being traversed with an endoscope. The tether 23 has a small cross sectional area so that it may pass through proximal seal 9, adjacent to endoscope without adversely affecting the function of the proximal seal 9. When the endoscope reaches the distal end of the guide tube 7, the tab on the tether 23 is removed. This allows the tether 23 to advance as the endoscope is advanced, thereby allowing the endoscope to force open the distal valve. The tether 23 can have a length significantly greater than the guide tube 7. In this case, when the tab is removed and the endoscope is advanced, there is still a section of tether 23 outside the body that the operator can control if they so choose. This extension may be attached when the tab is removed from the tether 23 or be an integral part of the tether 23.

The tether 23 may have various physical properties. In some embodiments, it can be flexible, and can have minimal elongation when subjected to normal loads of less than 10 pounds force. It may or may not have the ability to remain elongated under compressive force without buckling, or its length may have a combination of these attributes.

In one embodiment where the tether 23 has the ability resist compress forces at its distal end, the tether is withdrawn from the distal surface 36 of guide tube 7 prior to everting section of tube 7 where tether is secured. In one configuration shown in FIG. 15c, the distal end of the tether 23 is seated into a flexible tube 56 that is sealed at its proximal end. The tube 56 is fixed to the guide tube 7 at attachment point location consistent with points of attachment previously described for tether 23. Prior to the distal end of the guide tube everting, the tether is withdrawn from flexible tube 56.

In an alternative configuration, the proximal portion of the tether 23 resists compression and its distal end has low or nearly no compressive resistance properties. In this configuration the tether offers limited assistance pushing the distal end of the guide tube 7 while using lower than normal deployment pressure. The distal end of the tether 23 being very flexible does not significantly impede the eversion of the distal end of the guide tube 7. This configuration has the ability for the distal end of the guide tube 7 to be subsequently re-inverted to re-pressurize the lumen of the guide tube 7, while counter force is applied to the tether 23 as the endoscope is withdrawn from the guide tube 7 that remains stationary. In some embodiments, the compressive resistant tether 23 can be oriented in a straight configuration, and made from a creep resistant material so it does not take a curved shape when wound upon the deployment spool 6. Suitable material for the tether 23 includes spring wire, nitinol wire, and synthetics such as polyimide tube or solid core.

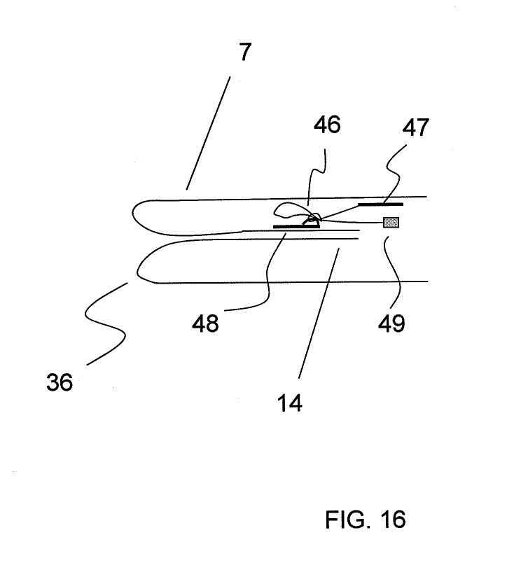

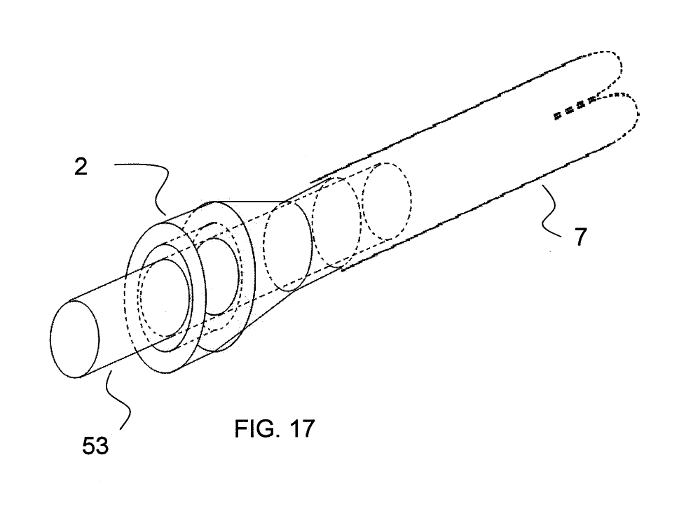

FIG. 16 shows another temporary valve formed by mechanically restraining the guide tube 7. This is accomplished by a tether 46 that temporarily anchors two points of the guide tube 7 at a point proximal to valve 14 as shown. The two anchor points are located at the distal end of the guide tube 7 and link the wall of the guide tube 7 at first anchor 48 to the wall of the guide tube at second anchor 47. First anchor 48 is located proximal of valve 14 and distal of second anchor 47. In some embodiments, the distance between the two anchor points when the guide tube 7 is straight is about at least twice the diameter of the guide tube 7. In other embodiments, the distance between the two anchor points is less than twice the diameter of the guide tube 7. In some embodiments, the tether 46 can be permanently secured at second anchor 47 and temporarily secured at first anchor 48 with a knot, such as a slip knot or other suitable knot that can be tied to a tether attachment portion on the first anchor 48. In some embodiments, the end of the tether 46 terminates in a small tab 49 having a length and width smaller than the diameter of the endoscope working channel. With the guide tube 7 deployed to the point where it is stopped by the restraint, it is internally pressurized while the endoscope is advanced to tab 49. The distal end of the guide tube 7 is opened by releasing first anchor 48 from the restriction of the tether 46 by, for example, pulling the tab 49 to undo the slip knot that fastens the tether 46 to the first anchor 48. In some embodiments, tab 49 is then drawn into the endoscope with an endoscopic grasper while the end of the endoscope provides counter force to anchor 48.

There are other means known to one skilled in the art to temporarily prevent the guide tube from fully everting during deployment. Examples of alternative means to accomplish this are a locking pin, locking catch, balloons, snares, pressure sensitive adhesion, banding, mechanical seals benefiting from internal pressure, diaphragms, etc.

An alternative means to advance the endoscope within a body lumen is to first deploy the guide tube 7 as previously described. With the guide tube 7 deployed within the body lumen, a semi-rigid guide is advanced down the entire length of the guide tube. The semi-rigid guide has a length greater than the guide tube 7 and a diameter less than the diameter of the working channel of an endoscope. With the semi-rigid guide in place, the guide tube is withdrawn from the body lumen while the semi-rigid guide remains fixed in place within the body lumen. An endoscope is now advanced within the body lumen over the semi-rigid guide.

As shown in FIG. 17 we disclose a means to accomplish an alternative means of straightening the proximal portion of a body lumen, especially the rectum and sigmoid colon to facilitate instrument passage. This is accomplished by placing a semi rigid tube within the proximal region of the colon by using the guide tube 7 as shown in FIG. 17. This embodiment uses a guide tube 7 that is deployed into the proximal colon as previously described here. Once the everting guide tube 7 is deployed, the everting guide tube 7 and insertion nozzle 2 are detached from the pressurization chamber 1 used for deployment. As shown in FIG. 17, a semi-rigid tube 53 is inserted through the deployment nozzle 2 and advanced into the everted guide tube 7 up to a physical stop located at the proximal end of the semi-rigid tube 53. The inside diameter of the guide tube 7 is larger than the outside diameter of the semi-rigid tube 53. The inside diameter of the semi-rigid tube 53 is larger than the diameter of the endoscope to be inserted. The manufacturing of the guide tube 7 and material are as previously discussed. The semi rigid tube 53 is made from a material such as silicone. The semi-rigid tube wall can have a separation that extends along its entire length. This separation facilitates removal of the semi-rigid tube 53 from the endoscope while leaving the endoscope in place.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. For example, features described in one embodiment can be used in another embodiment. The embodiments were chosen and described in order to best explain the principles of the invention and its practical application, to thereby enable others skilled in the art to best utilize the invention and various embodiments with various modifications as are suited to the particular use contemplated.

1-46. (canceled)

47. An endoscopic guide tube device to facilitate the insertion of an endoscope or similar device into a body lumen, the device comprising:
a pressurization chamber comprising a nozzle and an inner surface that defines an interior, wherein the nozzle has an opening;
an everting tube having a proximal end and a distal end, wherein the everting tube is mounted within the pressurization chamber with the proximal end of the everting tube attached to the pressurization chamber;
a port on the pressurization chamber configured to allow a positive pressure to be delivered to the interior of the pressurization chamber, wherein the positive pressure is configured to deploy the everting tube outwards through the nozzle;
and a tether having a proximal end and a distal end, wherein the distal end of tether is attached to one or more locations on a distal portion of the everting tube, wherein the proximal end of the tether is separately connected or held to an external body such that, prior to full deployment of the everting tube, the length of the distance between the two ends of the tether is less than the fully deployed length of the everting tube such that the tether prevents full deployment of the everting tube until the tether is released from the external body.

48. The device of claim 47, further comprising a distal seal located at the distal end of the everting tube.

49. The device of claim 48, wherein the distal seal comprises a viscous or adhesive material disposed within the distal end of the everting tube.

50. The device of claim 48, wherein the distal seal comprises a pressure bond at the distal end of the everting tube.

51. The device of claim 48, wherein the distal seal comprises an elastic or non-elastic material disposed around the distal end of the everting tube.

52. The device of claim 47, wherein the everting tube is wound on a rotatable deployment spool located within the pressurization chamber.

53. The device of claim 47, further comprising a deployment spool located within the pressurization chamber, wherein the proximal end of the tether passes though the nozzle of the pressurization chamber and is releasably attached to the deployment spool.

54. The device of claim 47, wherein the external body of the tether is dimensioned larger than either the inner diameter of the nozzle or the everting tube such that the external body is too large to pass through the nozzle.

55. The device of claim 47, wherein the pressurization chamber of the endoscopic guide tube device comprises a pressure relief valve configured to prevent excessive positive pressures from occurring within the everting tube.

56. The device of claim 47, wherein the pressurization chamber comprises a pressure seal configured to allow a body to be inserted into the everting tube while maintaining the positive pressure within the pressurization chamber.

57. The device of claim 47, wherein the nozzle has a proximal end, a distal end and a central region between the proximal end and the distal end, wherein the outside diameter of the central region of the nozzle is smaller than both the outside diameter at the proximal end and the outside diameter at the distal end of the nozzle.

58. The device of claim 47, wherein the surface of the everting tube is modified to reduce either its coefficient of friction or its adhesion properties, wherein the modification is selected from the group consisting of plasma energy treatment, application of polyvinylpyrrolidone, application of hyaluronic acid, application of parylene, application of friction reducing surface treatments, application of a biocompatible lubricating agent, application of glycerin, application of propylene glycol, application of a hydrophobic silicone based lubricant, and application of a water based lubricant.

59. The device of claim 47, wherein the distal end of the everting tube is temporarily sealed prior to deployment and wherein the distal end of the everting tube is configured to be opened by applying a force to the temporary seal at the distal end of the tube that is greater than the force used to cause the everting tube to deploy from the pressurization chamber.

60. The device of claim 47, wherein the distal end of the everting tube is closed and wherein the distal end of the everting tube is configured to be opened by cutting or puncturing or by application of mechanical force.

61. The device of claim 47, wherein the valve is formed from or attached to the distal end of the everting tube that has been modified such that, prior to full eversion, the distal end of the everting tube comprises two flat sections that are connected along two edges and have two opposing surface faces that are generally in direct contact with each other such that there is no significant gap between the two flat sections including at the edges where the two flat sections are connected such that a fluid barrier exists that prevents fluid from entering into the distal end of the everting tube.

62. The device of claim 61, wherein the valve is integral to the distal end of the everting tube.

63. The device of claim 47, wherein the port is configured to allow insertion of the endoscope and subsequent deployment of the everting tube by application of pressure from the endoscope.
